(19) United States
(12) Patent Application Publication
(10) Pub. No.: US 2025/0270385 A1
(43) Pub. Date: Aug. 28, 2025

(54) DYNAMICALLY CROSSLINKED MULTIBLOCK COPOLYMERS FOR COMPATIBILIZING IMMISCIBLE MIXED PLASTICS

(71) Applicants: COLORADO STATE UNIVERSITY RESEARCH FOUNDATION, Fort Collins, CO (US); THE TRUSTEES OF COLUMBIA UNIVERSITY IN THE CITY OF NEW YORK, New York, NY (US)

(72) Inventors: Eugene Y. CHEN, Fort Collins, CO (US); Ryan CLARKE, Fort Collins, CO (US); Kevin FRANKLIN, Fort Collins, CO (US); Tomislav ROVIS, New York, NY (US); Tobias SANDMEIER, Cambridge, MA (US); Dominik REICH, Adlkofen (DE)

(73) Assignees: COLORADO STATE UNIVERSITY RESEARCH FOUNDATION, Fort Collins, CO (US); THE TRUSTEES OF COLUMBIA UNIVERSITY IN THE CITY OF NEW YORK, New York, NY (US)

(21) Appl. No.: 18/856,955

(22) PCT Filed: Apr. 17, 2023
(86) PCT No.: PCT/US2023/018802

§ 371 Date: Oct. 15, 2024

Related U.S. Application Data
(60) Provisional application No. 63/332,140, filed on Apr. 18, 2022.
(60) Provisional application No. 63/332,197, filed on Apr. 18, 2022.

(51) Int. Cl. C08K 5/38; C08F 293/00; C08G 81/02; C08J 3/24; C08K 5/3442; C08K 5/372

(52) U.S. Cl. CPC C08K 5/38; C08F 293/005; C08G 81/027; C08J 3/246; C08K 5/3442; C08K 5/3725; C08J 2323/06; C08J 2367/04

(57) ABSTRACT

Described herein are compositions of matter for novel universal dynamic crosslinkers (UDCs) and the shortest closed-loop process for upcycling of any plastics, including post-consumer plastic waste and immiscible plastic mixtures enabled by UDCs. We discovered that the specifically designed UDCs can dynamically crosslink any plastics, and more importantly mixed immiscible plastics into compatibilized living grafted multiblock copolymers. Our studies show that such UDCs can reactivate mixed plastic dead chains and dynamically crosslink them into compatibilized multiblock copolymers. The in situ generated dynamic thermosets exhibit intrinsic reprocessability as well as enhanced tensile strength and creep resistance, relative to virgin plastics. This approach avoids the need for de/reconstruction and thus provides the maximum recovery of the endowed energy and materials value of the individual plastics.

### RELATED APPLICATIONS

This application claims priority under 35 U.S.C. § 119 (e) to U.S. Provisional Patent Application Nos. 63/332,140 and 63/332,197, both filed Apr. 18, 2022, which applications are incorporated herein by reference.

### GOVERNMENT SUPPORT

This invention was made with government support under grants DE-SC0022290 and DE-AC36-08GO28308 awarded by The US Department of Energy. The government has certain rights in the invention.

### BACKGROUND OF THE INVENTION

The global plastics problem is a trifecta, significantly impacting environment, energy, and climate. Many innovative closed/open-loop plastics recycling or upcycling strategies have been proposed or developed, addressing various aspects of the issues underpinning the achievement of a circular economy. In this context, reusing mixed plastics waste presents a particular challenge which has no current, effective closed-loop solution. This is because such mixed plastics, especially polar/apolar polymer mixtures, are typically incompatible and phase separate leading to materials with significantly inferior properties.

Leibler et al. (Science 2011, 334, 965) demonstrated that reversibly crosslinking a polymer to a vitrimer can yield a dynamic thermoset that can be repeatedly reprocessed while maintaining properties. Installation of dynamic and flexible crosslinkers has also been used to increase the adhesion of polymer melts onto polymeric surfaces. Another important concept introduced by Wulff et al. (Science 2019, 366, 875) is of universal static (permanent) crosslinkers (USCs) based on symmetric molecular bis(diazirine) precursors that, upon thermal or light activation, yield highly reactive bis(carbene) species that can crosslink seemingly any polymer bearing a C—H bond through inter-chain double C—H insertion of the carbene.

To achieve a circular plastics economy, one must overcome a central barrier of reusing mixed plastics waste because a significant fraction of plastics waste is in the form of mixed plastics. However, most polymer mixtures are immiscible, primarily because the entropy of mixing is dramatically reduced in these cases—in fact, this quantity scales inversely with the length of the chains, and for typical chains of length ˜1000, the entropy of mixing, which favors miscibility, is essentially zero. Thus, even a small energetic dislike between the segments of the two polymers readily yields an immiscible system. From a recycling viewpoint, there is thus a pressing need to compatibilize generally immiscible mixed plastic “dead” chains. It is important here to distinguish between miscibility and compatibilization. Miscibility requires homogeneity at the molecular level, a goal that may not be optimal given that the components of the mixed plastics have unique properties that we may wish to retain. In contrast, compatibilization ensures that the immiscible interface(s) between the distinct phases are reinforced by (macro) molecules that tie across them-surfactants are classic examples of such species. Thus, compatibilization ensures that the distinct phases retain their individual properties, while at the same time ensuring that the resulting material is thermomechanically robust.

To address the global problem of recycling of mixed plastics, strategies that can compatibilize polymer mixtures are needed to produce polymeric materials that can be easily recycled.

### SUMMARY

Mixtures of polar and apolar polymers are typically incompatible and phase separate leading to materials with significantly inferior properties. To address this barrier, here we introduce a compatibilization strategy that installs dynamic crosslinkers into several classes of binary, ternary, and “post-consumer” immiscible polymer mixtures in situ. Our combined experimental and modeling studies show that specifically designed classes of dynamic crosslinkers can reactivate mixed plastic “dead” chains, represented here by apolar polyolefins and polar polyesters, by compatibilizing them via dynamically forming graft multiblock copolymers. The resulting in-situ generated dynamic thermosets exhibit intrinsic reprocessability and enhanced tensile strength and creep resistance, relative to virgin plastics. This approach avoids the need for de/reconstruction and thus potentially provides an alternate, facile route towards the recovery of the endowed energy and materials value of the individual plastics.

Accordingly, this disclosure provides a crosslinking agent of Formula I:

R—X-L-G-L-X—R (I),

wherein,
each L is independently an aryl, heteroaryl, or alkylene;
G is anhydride, disulfide, thioester, ester, boronic ester, thioboronic ester, silyl ether, siloxane, imine, or urethane;
each R is independently alkyl, aryl or heteroaryl; and
X is C(N)₂ or carbene;
wherein each alkyl, alkylene, aryl, or heteroaryl is optionally substituted.

This disclosure also provides a dynamic thermoset that is crosslinked with the crosslinking agent described herein.

Additionally, this disclosure provides a method for crosslinking mixed plastics, comprising:
a) forming a mixture comprising apolar polymers, polar polymers, or a combination thereof;
b) blending the mixture and the crosslinking agent of any one of claims 1-9 to form a blend;
and c) activating the crosslinking agent;
   wherein the mixed plastics are thereby crosslinked.

The invention provides novel compounds (i.e., crosslinking agents) of Formulas I, II and IIB, intermediates for the synthesis of compounds of Formulas I, II and IIB, as well as methods of preparing compounds of Formulas I, II and IIB. The invention also provides compounds of Formulas I, II and IIB that are useful as intermediates for the synthesis of other useful compounds, polymers and copolymers.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C. Thermomechanical performance of crosslinked PE and UDC compatibilization of immiscible PE/polyester blends. a, Tensile stress/strain curves (5 mm/min, 25° C.) of LDPE and LDPE thermosets by UDC3 and USC, and reprocessed LDPE thermoset scraps, demonstrating recyclability of UDC-enabled thermosets and diminished recyclability of USC-based thermosets. b, Storage moduli overlay for HDPE, HDPE-UDC1, and HDPE-UDC3 highlighting crosslinking evidenced by rubbery plateau extension (5° C./min to fail, 0.3%, 1 Hz). c, Prolong-simulative creep-recovery performance for HDPE at 40° C. against PE-UDC1 and PE-UDC3 (2 MPa, 4 h creep, 30 min recovery).

FIG. 2A-D. Cross-sectional SEM images of LMWPE-PLLA blends. a, Virgin immiscible LMWPE-PLLA. b. Compatibilized LMWPE-PLLA blend processed with 5% UDC2. c, Un-compatibilized blend processed with 5% USC. d, Uncompatibilized blend processed with 5% MDC.

FIG. 3A-F. Cross-sectional SEM images of LMWPE-P3HB blends. a, Virgin immiscible LMWPE-P3HB. b, Compatibilized blend processed with UDC1. c, Compatibilized blend processed with UDC2. d, Compatibilized blend processed with UDC3. e, Uncompatibilized blend processed with USC. f, Uncompatibilized blend processed with MDC.

FIG. 4A-D. Reprocessability, recyclability, and UDC compatibilization of immiscible polyolefin/polyester binary and ternary blends. a, Melting temperatures for individual blend components of virgin LDPE-P3HB and blends crosslinked with UDC1-3 and USC (10° C./min). b, Storage moduli overlay plot for blends of virgin LDPE-P3HB and blends crosslinked with UDC1-3 and USC (5° C./min to fail, 0.3%, 1 Hz). c, Reprocessability/recyclability of the UDC-crosslinked LDPE-PLLA blend with tensile stress/strain tests (23° C., 5 mm/min) of specimens prepared by reactive extrusion and extrusion-reprocessed against virgin LDPE-PLLA blend. d, Melt extrusion reprocessability by DMA frequency sweeps on UDC-crosslinked LDPE-PLLA dynamic blends at first (blue) and second (red) cycle (5° C./min, 0.3%, 1 Hz).

FIG. 5A-D. Cross-sectional SEM images of LDPE-P3HB blends. a, Extruded immiscible virgin LDPE-P3HB. b, Compatibilized blend via reactive extrusion with 5% UDC1. c, Compatibilized blend via reactive extrusion with 5% UDC2. d, Compatibilized blend via reactive extrusion with 5% UDC3.

FIG. 6A-F. Cross-sectional SEM images of LDPE-PLLA blends. a, Extruded immiscible virgin LDPE-PLLA blend. b, Compatibilized blend via reactive extrusion with 5% UDC1. c, Compatibilized blend via reactive extrusion with 5% UDC2. d, Compatibilized blend via reactive extrusion with 5% UDC3. e, Extruded immiscible virgin blend of LDPE bag and PLLA cup flakes. f, Compatibilized blend of LDPE bag and PLLA cup flakes via reactive extrusion with 5% UDC3.

FIG. 7A-B. Cross-sectional SEM images of binary blends. a, Extruded immiscible virgin LDPE-iPP blend. b, Compatibilized LDPE-iPP blend via reactive extrusion with 5% UDC3.

FIG. 8A-B. Cross-sectional SEM images of binary blends. a, Extruded immiscible virgin PS-PLLA blend. b, Compatibilized PS-PLLA blend via reactive extrusion with 5% UDC3.

FIG. 9A-D. Sectional SEM images of ternary blends. a, Low-magnification image of extruded immiscible LDPE-iPP-PLLA ternary blend. b, High-magnification image of extruded immiscible LDPE-iPP-PLLA ternary blend. c, Low-magnification image of compatibilized LDPE-iPP-PLLA ternary blend via reactive extrusion with 5% UDC3. d, High-magnification image of compatibilized LDPE-iPP-PLLA ternary blend via reactive extrusion with 5% UDC3.

FIG. 10A-E. SEM images of the surfaces (left) and cross sections (right) (3 kV, 10 nm Au coating) of immiscible PE-PLLA blend and compatibilised PE-PLLA blend by UDC2 (5 wt %) (a). SEM cross-section images of PE-P3HB blends: virgin (b), dynamic UDC (c), static USC (d), and mono-diazirine control MDC (e). See also FIG. 1A-C.

FIG. 11A-E. Cross-sectional SEM images revealing degree of UDC compatibilisation by droplet analysis for immiscible LDPE-PLLA virgin blend (a) and UDC3-compatibilised LDPE-PLLA blend (b). SEM cross-sectional images revealing degree of UDC compatibilisation of immiscible ternary LDPE-iPP-PLLA virgin blend (c) and UDC3-compatibilised LDPE-iPP-PLLA blend (d). Compatibilisation of real-world mixed-plastics: photograph of LDPE bag and PLLA cup (i), subsequent flakes by mechanical shredding for melt-extrusion highlighting included colourants and additives (ii), and cross-sectional SEM images revealing degree of UDC compatibilisation of the immiscible LDPE bag-PLLA cup (top) blend and the UDC3-compatibilised LDPE bag-PLLA cup blend (bottom) (iii) (e). See also FIG. 4A-D.

### DETAILED DESCRIPTION

Described herein are “living” grafted multiblock copolymers (gMBCPs) that can be formed when a specifically designed universal dynamic crosslinkers (UDCs) is added to an immiscible polymer mixture, to compatibilise the polymer mixture into reprocessable and recyclable dynamic thermosets. In contrast to the USC, such UDCs incorporate the dynamic nature of vitrimers into the universal crosslinking of polymer mixtures to achieve the holy grails of compatibility coupled with the ability to have multiple use cycles (Chart 1).

Chart 1. Design of universal dynamic crosslinkers (UDCs). (A) Comparisons between the known bis(diazirine)-based universal static crosslinker (USC) scaffold and redesigned UDCs 1-3 embedded with dynamically exchangeable bonds between the crosslinking sites. (B) Generalized UDC structural compositions:

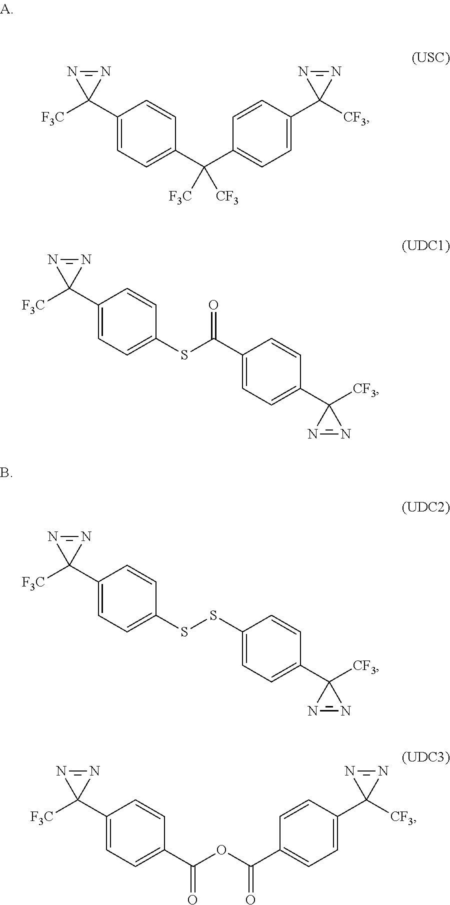 [SMILES: FC(F)(F)C1(c2ccc(C(c3ccc(C4(C(F)(F)F)N=N4)cc3)(C(F)(F)F)C(F)(F)F)cc2)N=N1 ; O=C(Sc1ccc(C2(C(F)(F)F)N=N2)cc1)c1ccc(C2(C(F)(F)F)N=N2)cc1 ; FC(F)(F)C1(c2ccc(SSc3ccc(C4(C(F)(F)F)N=N4)cc3)cc2)N=N1 ; O=C(OC(=O)c1ccc(C2(C(F)(F)F)N=N2)cc1)c1ccc(C2(C(F)(F)F)N=N2)cc1]

Reactive species that can insert into C—H bonds, e.g., carbene or precursors that can form carbenes by external stimuli.

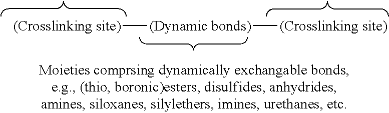

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units is also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2, 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution or solid state, in a reaction mixture.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms, or an alkenylene can have the two free valences on the same carbon.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents R^{X} where R^{X} is at the ortho-, meta-, or para-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′) C(O)R′, N(R′)N(R′) C(O)OR′, N(R′)N(R′)CON(R′)₂, N(R′)SO₂R′, N(R′)SO₂N(R′)₂, N(R′) C(O)OR′, N(R′) C(O)R′, N(R′) C(S)R′, N(R′) C(O)N(R′)₂, N(R′) C(S)N(R′)₂, N(COR′) COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

A “solvent” as described herein can include water or an organic solvent. Examples of organic solvents include hydrocarbons such as toluene, xylene, hexane, and heptane; chlorinated solvents such as methylene chloride, chloroform, and dichloroethane; ethers such as diethyl ether, tetrahydrofuran, and dibutyl ether; ketones such as acetone and 2-butanone; esters such as ethyl acetate and butyl acetate; nitriles such as acetonitrile; alcohols such as methanol, ethanol, and tert-butanol; and aprotic polar solvents such as N,N-dimethylformamide (DMF), N,N-dimethylacetamide (DMA), and dimethyl sulfoxide (DMSO). Solvents may be used alone or two or more of them may be mixed for use to provide a “solvent system”.

A non-polar solvent is a liquid or solvent that has a low or non-existing dipole moment and is missing any partial positive or negative charges. Generally, it has small differences in electronegativity between atoms in the solvent molecule and has a low dielectric constant. A non-polar solvent cannot effectively dissolve a polar compound. Examples of a non-polar solvent includes alkanes, toluene, chloroform and diethyl ether.

The term, “repeat unit”, “repeating unit”, or “block” as used herein refers to the moiety of a polymer that is repetitive. The repeat unit may comprise one or more repeat units, labeled as, for example, repeat unit A, repeat unit B, repeat unit C, etc. Repeat units A-C, for example, may be covalently bound together to form a combined repeat unit. Monomers or a combination of one or more different monomers can be combined to form a (combined) repeat unit of a polymer or copolymer.

The term “molecular weight” for the copolymers disclosed herein refers to the average number molecular weight (Mₙ). The corresponding weight average molecular weight (M_{w}) can be determined from other disclosed parameters by methods (e.g., by calculation) known to the skilled artisan.

The phrase “dynamically exchangeable bond” refers to a moiety that comprises bonds that can break in response to external stimuli, such as heat, light, or pH, and reform or self-heal to its original state or a different state. Dynamic bonds introduce properties such as self-healing, recyclability, shape memory, and malleability to polymers (see Angew. Chem. Int. Ed. 2022, 61, e202206938). Examples of moieties that have dynamically exchangeable bonds include anhydrides, disulfides, thioesters, esters, boronic esters, thioboronic esters, silyl ethers, siloxanes, imines, and/or urethanes.

### Embodiments of the Technology

1. A crosslinking agent of Formula I:

R—X-L-G-L-X—R (I),
wherein,
   each L is independently an aryl, heteroaryl, alkylene, alkenylene, or alkynylene;
   G is a moiety that comprises a dynamically exchangeable bond, wherein the moiety is an anhydride, disulfide, thioester, ester, boronic ester, thioboronic ester, silyl ether, siloxane, imine, or urethane, or another dynamically exchangeable bond moiety known to those of skill in the art;
   each R is independently alkyl, aryl, heteroaryl, or H; and
   X is C(N)₂ or carbene;
wherein each alkyl, alkylene, alkenylene, aryl, or heteroaryl is optionally substituted.
   L is generally defined as linking group or linker. In some embodiments, L is each independently a 6-membered aryl or 6-membered heteroaryl group. In general, R can be any group that is compatible with a carbene. In some other embodiments, R is each independently H, halo, or —(C₁-C₆)alkyl substituted with halo, wherein the number of halo substituents is 3, 1, 2, or 4 to 13.
2. The crosslinking agent of embodiment 1 wherein L is phenyl or pyridyl.
3. The crosslinking agent of embodiments 1 or 2 wherein X is in the para-position relative to G.
4. The crosslinking agent of any one of embodiments 1-3 wherein R is CF₃.
5. The crosslinking agent of any one of embodiments 1˜4 wherein X is

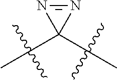 [SMILES: *C1(*)N=N1]

or —C═N⁺═N⁻.
6. The crosslinking agent of any one of embodiments 1-5 wherein G is —(C═O)O(O═C)—, —S—S—, —S(C═O)—, —OB(R)O—, or —OSi(R₂)O—.
7. The crosslinking agent of any one of embodiments 1-6 wherein each R—X-L- group consists of the same moieties.
8. The crosslinking agent of any one of embodiments 1-7 wherein Formula I is represented by Formula II:

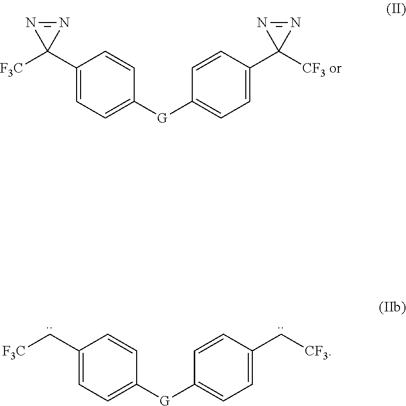

9. The crosslinking agent of embodiment 1 wherein the crosslinking agent is:

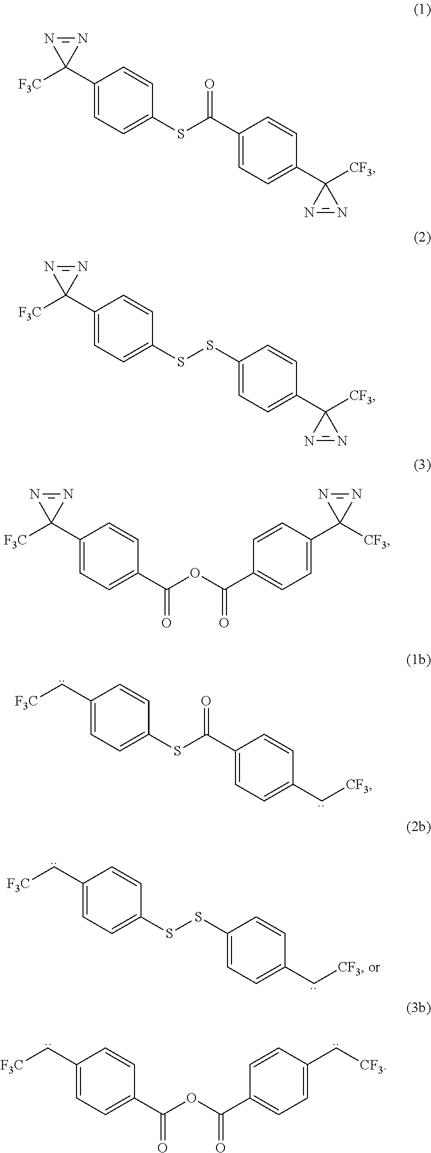

10. A dynamic thermoset that is crosslinked with the crosslinking agent of any one of embodiments 1-9. In some embodiments, the dynamic thermoset is a dynamically crosslinked thermoset.
11. The dynamic thermoset of embodiment 10 wherein the dynamic thermoset comprises a crosslinker in an amount of about 0.05 wt. % to about 50 wt. %, or about 1 wt. % to about 15 wt. %, wherein the crosslinker is a moiety of the crosslinking agent that is covalently bonded at its carbene carbon atoms (i.e., the carbon atom of X of Formula I, wherein X is CH) to polymers or copolymers in the dynamic thermoset.

In some other embodiments, the amount of the crosslinker is about 0.1 wt. %, about 0.5 wt. %, about 1 wt. %, about 1.5 wt. %, about 2 wt. %, about 3 wt. %, about 4 wt. %, about 5 wt. %, about 10 wt. %, about 15 wt. %, about 20 wt. %, about 30 wt. %, about 40 wt. %, or about 50 wt. %.
12. The dynamic thermoset of embodiment 10 or 11 wherein the dynamic thermoset comprises an apolar polyolefin, a polar polyester, or a combination thereof.
13. The dynamic thermoset of any one of embodiments 10-12 wherein the dynamic thermoset comprises low-molecular-weight polyethylene (LMWPE), low-density polyethylene (LDPE), high-density polyethylene (HDPE), ultra-high-molecular-weight polyethylene (UHMWPE), isotactic polypropylene (ifPP), polystyrene (PS), poly(L-lactic acid) (PLLA), poly(3-hydroxybutyrate) (P3HB), or a combination thereof.
14. A method for crosslinking mixed plastics, comprising:
   a) forming a mixture comprising apolar polymers, polar polymers, or a combination thereof;
   b) blending the mixture and the crosslinking agent of any one of embodiments 1-9 to form a blend;
   c) optionally drying the blend; and
   d) activating the crosslinking agent; wherein the mixed plastics are thereby crosslinked.

In some embodiments, the mixture is a mixture of solids, oils, liquids, or a combination thereof. In some embodiments, activating is induced thermally, chemically, physically, by pressure, or by irradiation. In some other embodiments, activating is induced reactive melt extrusion, such as in a twin-screw extruder.
15. The method of embodiment 14 wherein the mixture is blended with about 1 wt. % to about 15 wt. % of the crosslinking agent; or about 2 wt. %, about 3 wt. %, about 4 wt. %, or about 5 wt. % of the crosslinking agent; wherein the wt. % is based on the combined weight of the apolar polymer and the polar polymer.

### Results and Discussion

The USC-derived permanent network architecture is responsible for enhancing materials performance but deprives the medium to high-molecular-weight polymers of end-of-life reprocessability. We reasoned that synergistic coupling of USCs and vitrimers by installing dynamic crosslinks commonly found in covalent adaptable networks (CANs) into bis(diazirine) universal crosslinkers should yield UDCs capable of exchange reactions within a polymer matrix. This design should enable the effective reconfiguration of network topology, thus allowing the thermoset to access flow-state reprocessing upon exposure to exchange-activating stimuli, while maintaining the desirable thermoset material advantages within the parameterized working conditions. Exchange pathways and crosslink motifs have widely expanded to adapt available functionality in various polymer archetypes, including ubiquitous moieties such as esters, thioesters, boronic esters, disulfides, acids and anhydrides and silyl ethers. Ultimately, merging these two advances in the form of UDCs should enable an optimal strategy for upcycling any mixed plastics by dynamically crosslinking them into value-added thermosets while installing their reprocessability and compatibility.

### Universal Dynamic Crosslinkers (UDCs).

The initial development of UDCs required careful selection of dynamic linkers within the bis(diazirine) framework. They must be capable of dynamic exchange when stimulated, which is crucial for recyclability, and their potential reaction to the adjacent carbenes during crosslinking must be insignificant. UDCs 1-3 were prudently equipped with thioester, disulfide, and anhydride moieties, operating on catalyzed thiolate-thioester, disulfide metathesis, and anhydride exchange, respectively. We envisioned a convergent synthetic approach to these UDCs using 4-(3-(trifluoromethyl)-3H-diazirin-3-yl)benzenethiol (TDBT) and commercial 4-(3-(trifluoromethyl)-3H-diazirin-3-yl)benzoic acid (TDBA) as key intermediates. TDBT was readily accessed in a multi-step route from commercial materials, allowing preparation on multi-gram scale. The simplicity of UDC derivatization offers scalability and potentially broad access for various undiscovered dynamic linkers. Each UDC derivative exhibits UV-region absorption bands at up to ca. 390 nm characteristic of the bis(diazirine) functionality. Diazirine activation for carbene delivery was monitored through the mass % loss of two N₂ molecules at elevated temperatures by thermogravimetric analysis (TGA). Exotherms further signaling successful carbene formation (113-122° C.) during differential scanning calorimetry (DSC) scans were deemed non-explosive and non-shock sensitive.

Establishing high crosslinking efficacy without damaging the dynamic linker group is imperative for any UDC derivative prior to polymer application. Cyclohexane was chosen as the model compound to enable isolation and full characterization of crosslinked products. Thermal activation of UDC1-3 outperformed photochemical alternatives, inserting in up to 53% yield, which is notably higher than that observed in the USC, likely due to subtle changes in electron density of the resulting trifluoro aryl carbenes as more electron-rich diazirines have been shown to result in higher C—H insertion yields. Collectively, our experiments demonstrate that UDC1-3 can readily crosslink aliphatic substrates without significant degradation during carbene insertion. Additional control experiments revealed the crosslinking procedure is air and moisture tolerant, simplifying polymer application protocols. Next, cyclohexane and methyl methoxyacetate were chosen as small-molecule models for apolar polyolefins and polar polyesters, respectively (Scheme 1). A mixture of the model molecules and UDC2 was incubated at 130° C. and the hetero-crosslinked product (CM) was confirmed by high-resolution mass spectrometry. More detailed analysis by supercritical fluid chromatography (SFC) further revealed that formation of the mixed-crosslinked CM product is competitive with homo-crosslinking (CC and MM). In addition, heating a mixture of the two homocoupling products CC and MM furnished the mixed product CM, thus providing evidence for a thermal disulfide metathesis mechanism (Scheme 1). Furthermore, methyl propionate was used as a small molecule model substrate to probe the regioselectivity of C—H insertion into polyesters. After incubation at 130° C., insertion into C—H bonds a to the carbonyl was found predominantly.

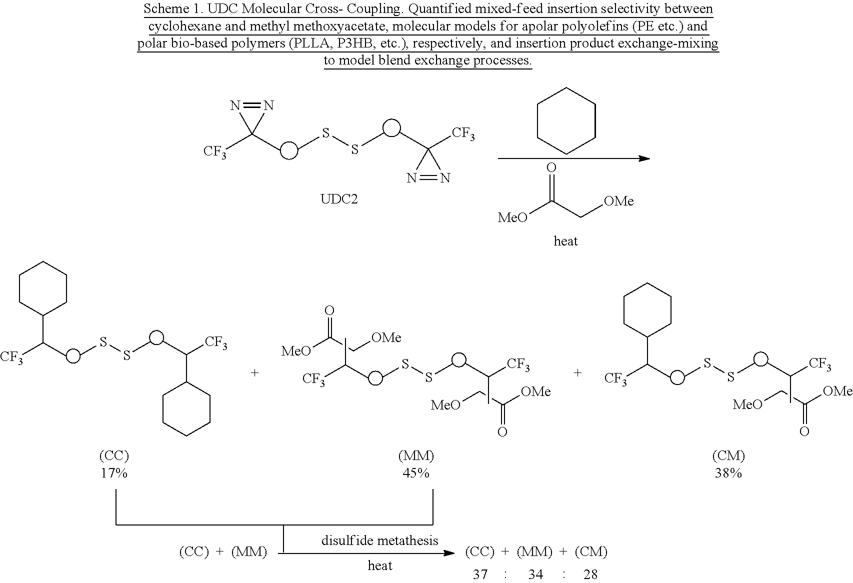

### Dynamic Crosslinking of Pure Thermoplastics to Reprocessable Thermosets.

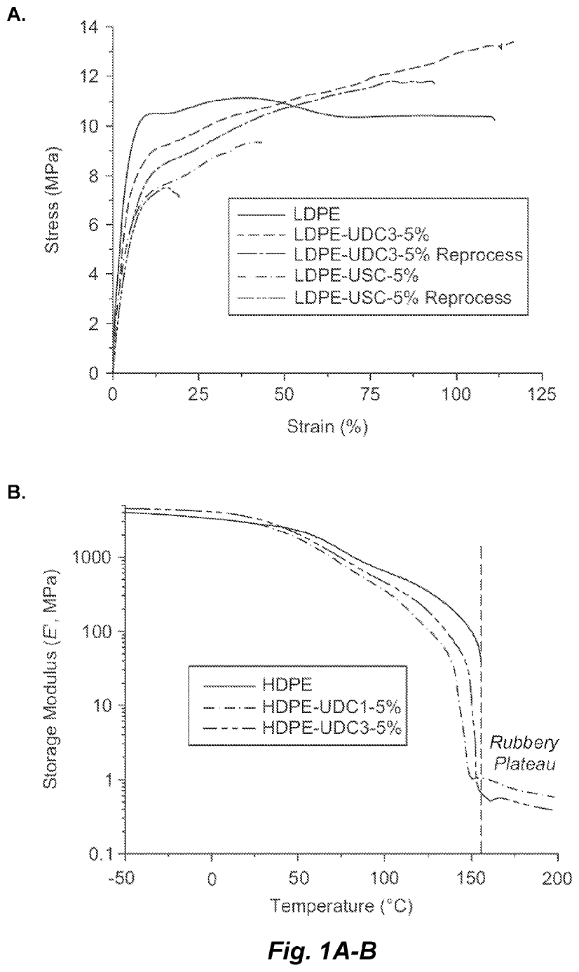

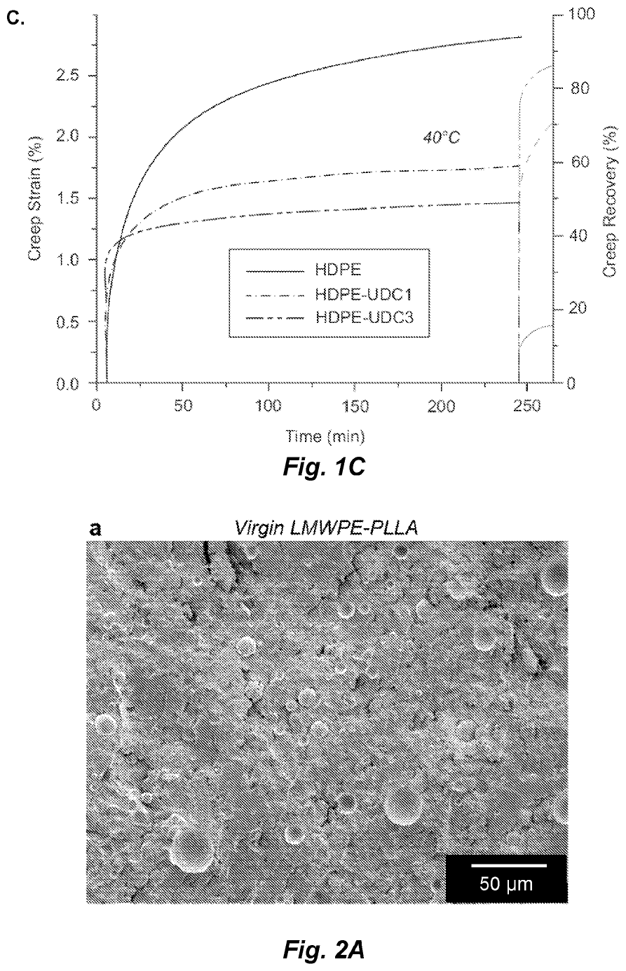

To establish the crosslinking of relevant thermoplastics, low-molecular-weight polyethylene (LMWPE), low-density PE (LDPE), high-density PE (HDPE), ultra-high-molecular-weight PE, and isotactic polypropylene (iPP), were treated with 0, 5, and 10% (by weight) UDC1-3. Reduced enthalpy of fusion and melting temperature (Tₘ) of a semicrystalline thermoplastic verified inserting crosslinks in the melt state. In contrast, the glass-transition temperature (T_{g}) increases on crosslinking. Crosslinking success was also supported by T_{g} increases in amorphous or semicrystalline samples, complemented by substantially reduced or eliminated solubility. Loadings of 5% UDC1-3 were selected for scaling, striking a balance between the UDC equivalent and the extent of mixing with polymers. FTIR analysis of melt-processed thin films of the model LMWPE substrate, pre- and post-UDC treatment, showed no change in polymer structure, mitigating decomposition concerns. Comparative tensile tests were next performed on LDPE [melt flow index (MFI)=21], a suitable candidate for homogeneous crosslinker impregnation as the virgin material achieves full solubility in non-reactive solvent at temperatures safely below the carbene activation temperature (Table 1). UDC3 was selected and compared against the static USC as both crosslinkers operate in the absence of external additives or catalysts. Embedding LDPE with UDC3 resulted in films with favorable mechanical performance relative to virgin LDPE, with increased strength at break (13 vs. 11 MPa) and tensile toughness (13 vs. 12 MJ m⁻³) but the same elongation at break (˜115%) (FIG. 1a). LDPE thermosets prepared by USC were inferior in strength (8.9 MPa), ductility (38%), and toughness (3.0 MJ m⁻³) relative to the virgin and UDC-treated LDPE (FIG. 1a). Further distinctions between UDC and USC are emphasized by reprocessing experiments, in which LDPE-UDC3 exhibited a similarly competitive tensile profile before and after reprocessing, while LDPE-USC lacked reprocessability after single reprocessing, yielding drastic decreases in strength, strain, and toughness by 41%, 82%, and 88%, respectively (FIG. 1a, Table 1).

TABLE 1
Tensile stress/strain data for each reported LDPE-UDC and
LDPE-USC sample, along with reprocessing trial data.

| | Stress | Strain | Modulus | Toughness |
|---|---|---|---|---|
| Polymer | (MPa) | (%) | (MPa) | (MJ m⁻³) |
| LDPE | 10.5 ± 0.1 | 113 ± 5.8 | 345 ± 14 | 12.4 ± 0.7 |
| LDPE 5% UDC3 | 13 ± 0.4 | 116 ± 4.0 | 245 ± 3.0 | 12.7 ± 0.6 |
| LDPE 5% UDC3 | 12 ± 0.4 | 91 ± 6.6 | 166 ± 16 | 8.9 ± 0.5 |
| R1 | | | | |
| LDPE 5% USC | 8.9 ± 1.0 | 38 ± 12 | 166 ± 10 | 2.8 ± 1.1 |
| LDPE 5% USC | 7.5 ± 0.5 | 16 ± 3.7 | 160 ± 6.4 | 1.0 ± 0.3 |
| R1 | | | | |

Melt-pressed films of UDC1- and 3-embedded LDPE and HDPE (MFI=7.6) retained or improved their storage modulus (E′) values at both −50° C. and 23° C., respectively, relative to the virgin materials. A maximum E′ of 3.2 GPa for LDPE and 4.2 GPa for HDPE were obtained when crosslinked with UDC3; these numbers are noticeably higher than the thermosets crosslinked with UDC1 (FIG. 1b, Table 2). We attribute the superior thermomechanical performance of UDC3-crosslinked materials to the stability of the anhydride dynamic linker, which is uncatalyzed and less prone to ambient creep than those derived from UDC1. Thermal frequency sweeps revealed the presence of a rubbery plateau (E′˜constant) above Tₘ observed in crosslinked samples, indicating practically advantageous thermomechanical durability of the crosslinked PE materials relative to non-crosslinked analogs (FIG. 1b). For example, at 150° C. the UDC crosslinked HDPE samples have E′˜0.3-1 MPa, depending on the UDC. The non-crosslinked samples fail under these conditions. The plateau modulus of HDPE melts, which is attributable primarily to entanglements, is ˜1 MPa. Thus, we conclude that the dynamic crosslinks provided by the UDC occur at length scales comparable to the native entanglements but temporally are more persistent and thus provide a measurable modulus. Since it is known that the mean distance between entanglements along the chain contour is ˜50 monomers, this implies from rubber elasticity that the UDC systems correspond to networks with an elastically active crosslink fraction of 1 vol %. This analysis suggested that only a small fraction (˜20%) of the UDC molecules are elastically effective—this could either be due to the high temperature of the measurement, or the likelihood that some crosslinks are intramolecular and hence elastically inactive.

TABLE 2
Temperature-ramp frequency sweep results for
virgin and UDC-treated LDPE/HDPE samples (0.3%
strain, 1 Hz, −50° C. to 200° C., 5° C./min).

| Polymer | E′_{RT} (MPa) | E′ₘₐₓ (MPa) |
|---|---|---|
| LDPE 0% | 784 ± 149 | 2531 ± 208 |
| LDPE 5% UDC1 | 730 ± 92 | 3055 ± 157 |
| LDPE 5% UDC3 | 977 ± 110 | 3198 ± 12 |
| HDPE 0% | 2990 ± 74 | 3818 ± 138 |
| HDPE 5% UDC1 | 2840 ± 105 | 3941 ± 72 |
| HDPE 5% UDC3 | 3127 ± 158 | 4224 ± 188 |

To further demonstrate that the UDC technology can bridge thermoset performance and thermoplastic recyclability, we investigated the creep-recovery behavior of crosslinked LDPE and HDPE below their Tₘ (by ˜30° C. each) and well-within film operating conditions (Table 3). Virgin-quality LDPE exhibited the most pronounced creep deformation (25.5%) over the course of 10 min at 80° C. in comparison to LDPE reinforced with UDC1 (9.0%) and UDC3 (12.4%), while suffering nearly 30% permanent deformation upon load removal. Similarly, HDPE films treated with UDC1 and UDC3 improved both creep resistance (2.6% and 1.7% less, respectively) and recovery (+˜30%) relative to the untreated HDPE film at 110° C. Impressively, in a 4-h creep experiment mimicking working conditions at 40° C. and prolonged stress (2 MPa), the creep deformation of HDPE was nearly halved with UDC1 and UDC3 incorporation (1.47% and 1.76%, respectively, vs. 2.82%) (FIG. 1c). Recovered strain from the 4-h load event was significantly improved from 15% to over 70% in both HDPE-UDC specimens, displaying an added elastic character unattainable by HDPE chain entanglement alone.

TABLE 3
Creep-recovery data for virgin and UDC-treated LDPE and HDPE
by DMA, in tension film mode, below the melt thermal transition
(40-110° C., 2 MPa, 5° C./min 600 s or 4 h as specified
creep duration, 300 s or 0.5 h as specified recovery time).

| | | Creep/ | | |
|---|---|---|---|---|
| | Temperature | Recovery | | Recovered |
| Sample | (° C.) | (min) | Creep(%) | (%) |
| LDPE (MFI 21) | 80 | 10/5 | 26.5 | 19.1 |
| HDPE (MFI 7.6) | 110 | 10/5 | 7.4 | 4.7 |
| HDPE (MFI 7.6) | 40 | 240/30 | 2.8 | 0.4 |
| LDPE UDC1 5% | 80 | 10/5 | 12.4 | 11.7 |
| LDPE UDC3 5% | 80 | 10/5 | 9.0 | 8.1 |
| HDPE UDC1 5% | 110 | 10/5 | 4.8 | 4.2 |
| HDPE UDC3 5% | 110 | 10/5 | 5.7 | 5.1 |
| HDPE UDC1 5% | 40 | 240/30 | 1.8 | 1.3 |
| HDPE UDC3 5% | 40 | 240/30 | 1.5 | 1.3 |

Flow-state mobility and paralleling reconfigurability behavior of UDC crosslinked PEs were characterized by bulk shear oscillatory rheology. Creep-recovery was performed on LDPE at 130 and 140° C. as well as HDPE at 150 and 160° C., 15 and 25° C. above the respective Tₘ values (Table 4). As expected, virgin LDPE and HDPE thermoplastics experienced extreme creep in the melt state. In comparison, thermosets prepared with UDC1 and 3 exhibited significantly reduced creep consistent with a viscous material highly influenced by both temperature and the dynamic linker exchange pathway. We also demonstrated the lack of melt configurability of the USC crosslinked LDPE thermoset by analyzing the creep at 130° C. and found the least amount of shear strain with nearly full recovery upon removal of shear stress. Overall, the demonstrated orthogonality-creep suppression below the Tₘ and retained creep above the Tₘ—is key to the recycling advantages of UDC-crosslinked materials.

TABLE 4
Creep-recovery data for virgin, USC-, and UDC-treated
LDPE and HDPE by bulk shear rheology in the flow-state
(130-160° C., 10 min creep, 5 min recovery, 2.5 kPa).

| Sample | Temperature (° C.) | Creep (%) | Recovered (%) |
|---|---|---|---|
| LDPE (MFI 21) | 130 | 90877 | 112 |
| HDPE (MFI 7.6) | 150 | 77681 | 28 |
| LDPE UOC1 5% | 130 | 53 ± 8.0 | 22 ± 1.1 |
| LDPE UDC1 5% | 140 | 103 ± 7.5 | 66 ± 0.6 |
| LDPE UDC3 5% | 130 | 89 ± 34 | 41 ± 1.2 |
| LDPE UDC3 5% | 140 | 190 ± 33 | 40 ± 1.6 |
| HDPE UDC1 5% | 150 | 411 ± 16 | 25 ± 0.3 |
| HDPE UDC1 5% | 160 | 610 ± 20 | 31 ± 1.4 |
| HDPE UDC3 5% | 150 | 521 ± 25 | 73 ± 14 |
| HDPE UDC3 5% | 160 | 808 ± 19 | 207 ± 7.5 |
| LDPE USC 5% | 130 | 24 ± 1.6 | 21 ± 1.1 |

### Compatibilization of Immiscible Polyolefin/Polyester Blends.

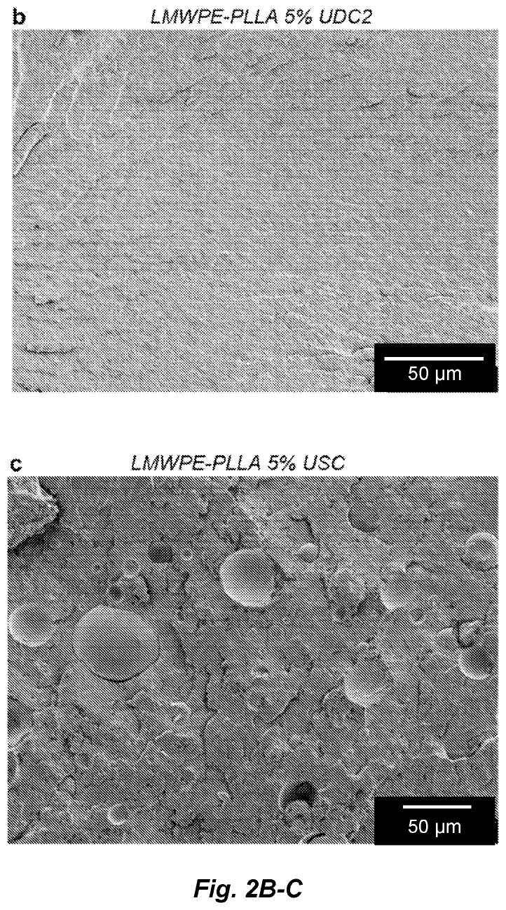

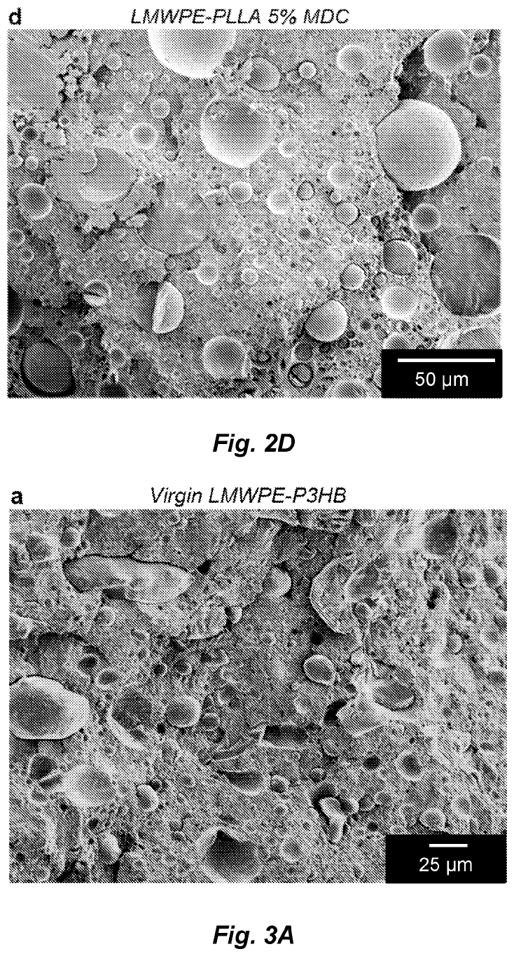

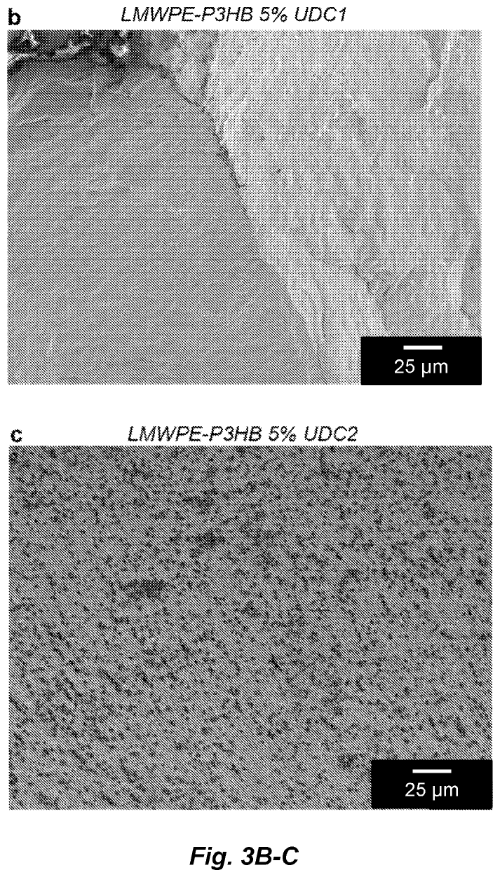

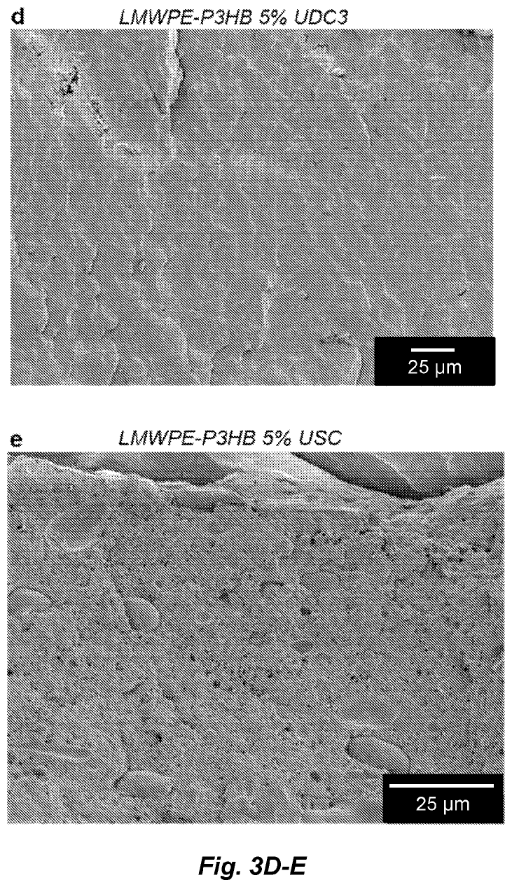

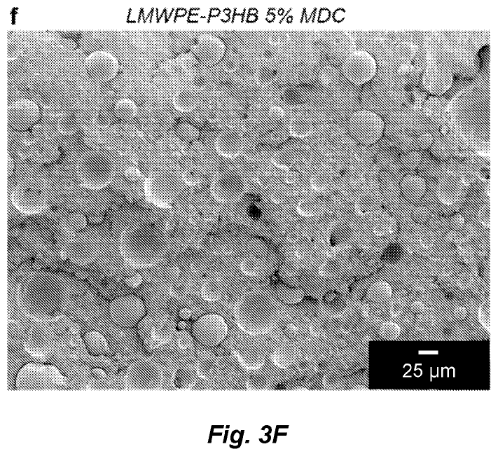

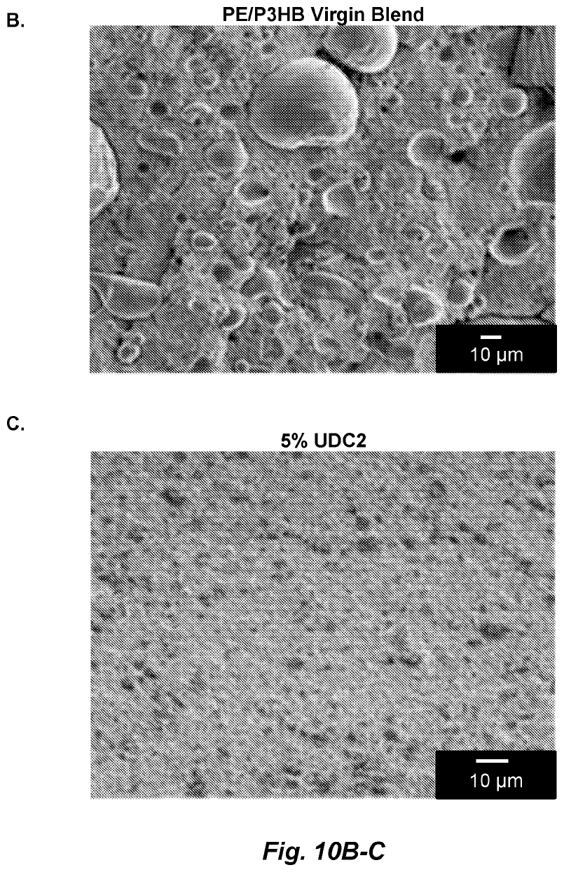

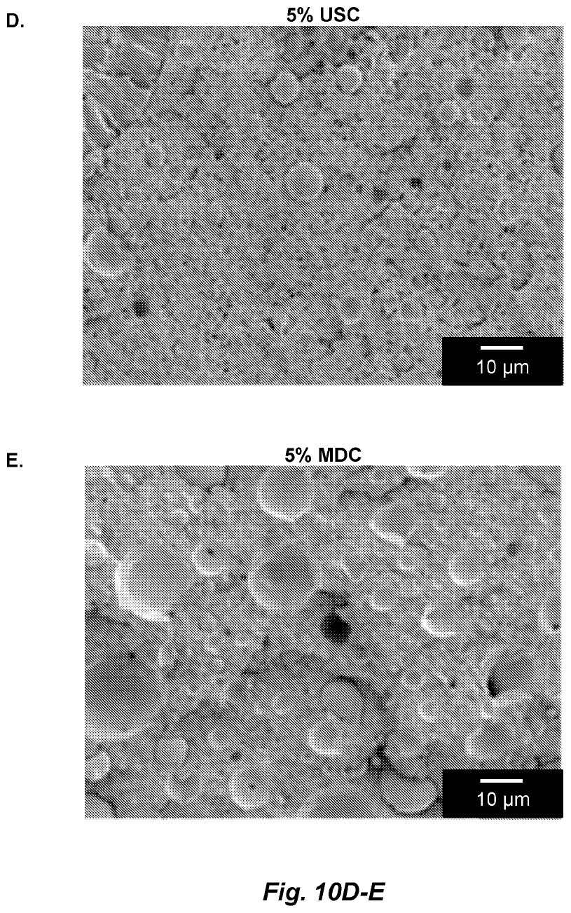

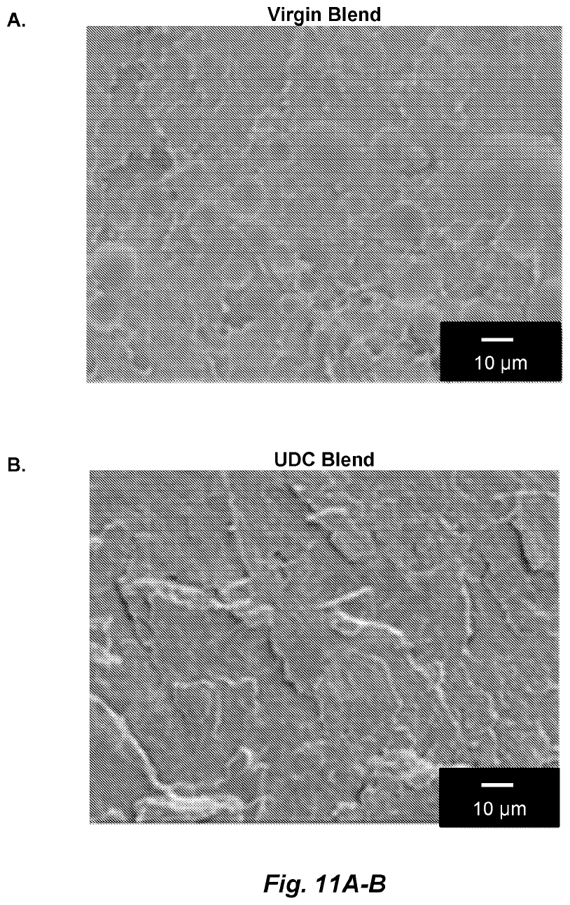

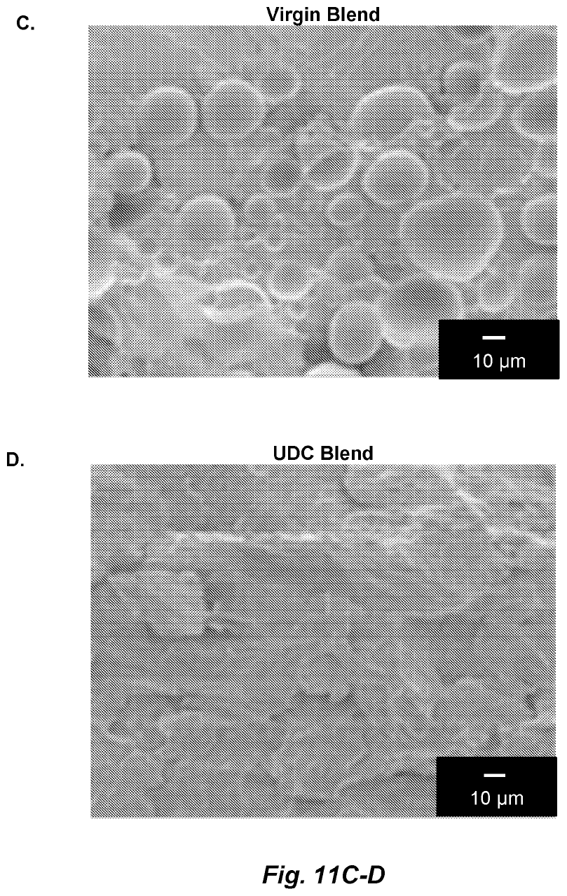

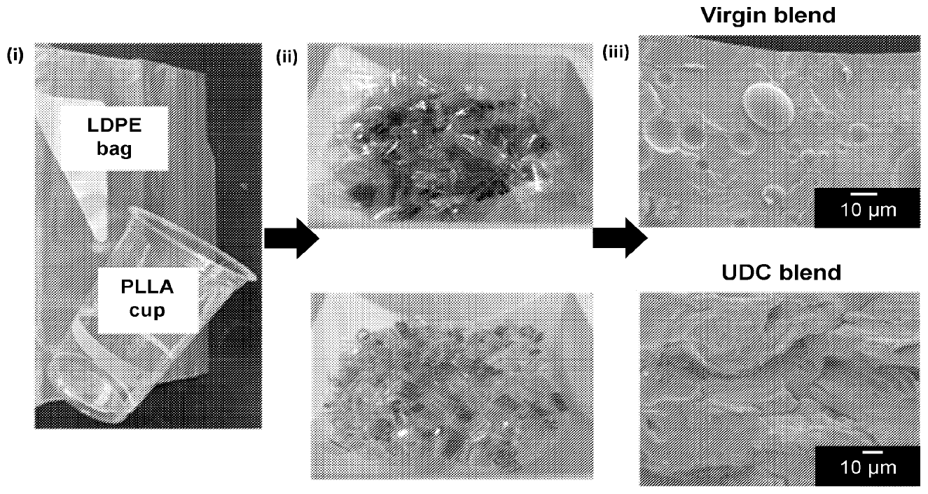

Nearly all postconsumer plastics exist in mixed-waste streams. Thus, we considered the creation of compatibilized dynamic thermosets by blending incompatible polymer archetypes with the UDCs (Chart 2). Common bio-based poly(L-lactic acid) (PLLA) and poly(3-hydroxybutyrate) (P3HB) were selected for blending trials with LMWPE and LDPE. Average droplet diameter size, tracked by scanning electron microscopy (SEM), serves as the first metric for UDC compatibilization efficacy (Table 5, Table 6). As expected, the incompatible LMWPE-PLLA and LMWPE-P3HB blends of heterogeneous films revealed large PE-polyester domain interfaces with large droplets averaging 29 μm (PLLA) and 32 μm (P3HB) in diameter, respectively (FIG. 2a, FIG. 3a and FIG. 10a-e). Static crosslinking by USC results in the continued presence of droplets, though their average diameters are reduced to 2.1 μm (PLLA) and 1.6 μm (P3HB), respectively (FIG. 2c and FIG. 3e, Table 5). However, the incorporation of UDC2 or 3 (5%) to either of the blends during mixing improved surface homogeneity so drastically that droplet formation was not observed (FIG. 2b and FIG. 3c, 3d), strongly suggesting compatibilized blends. In the case of UDC1, droplets still formed but the average droplet diameter was substantially reduced to ˜2.8 μm (FIG. 3b). In contrast to the bis(diazirine) counterparts, MDC (a linker with only one functionality) increased the average droplet diameter to 45 μm (PLLA) and 48 μm (P3HB), respectively (FIG. 2d and FIG. 3f).

### Chart 2. Apolar and Polar Polymers.

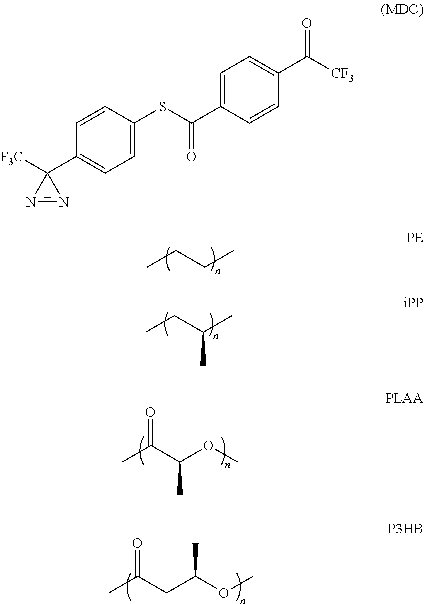

TABLE 5
Average diameter (micrometers) and median diameter (micrometers)
for droplets imaged within the cross-sections of LMWPE-
PLA/P3HB binary blends both of virgin quality and once
crosslinked with UDCs 1-3, USC, or functionalized with
MDC as described (10 micrometers gold, 3 kV).

| | Average | Median | |
|---|---|---|---|
| Blend Sample | Diameter (μm) | Diameter (μm) | Notes |
| Virgin PE-PLA | 29 ± 11 | 29 | Minority smaller |
| | | | droplets also found |
| PE-PLA UDC2 | — | — | No droplets found |
| PE-PLA USC | 2.1 ± 0.9 | 2.0 | Minority larger |
| | | | droplets also found |
| PE-PLA MDC | 45 ± 23 | 43 | Minority smaller |
| | | | droplets also found |
| Virgin PE-P3HB | 32 ± 13 | 25 | Minority smaller |
| | | | droplets also found |
| PE-P3HB UDC1 | 2.8 ± 1.4 | 2.7 | Low droplet |
| | | | population |
| PE-P3HB UDC2 | — | — | No droplets found |
| PE-P3HB UDC3 | — | — | No droplets found |
| PE-P3HB USC | 1.6 ± 0.7 | 1.8 | Minority smaller |
| | | | droplets also found |
| PE-P3HB MDC | 48 ± 17 | 47 | Minority larger |
| | | | droplets also found |

These microscopy measurements are complemented by small- and wide-angle X-ray scattering (SAXS and WAXS) analysis, revealing that the virgin and UDC-compatibilized blends of LDPE-P3HB and LDPE-PLLA have WAXS patterns that are linear superpositions of the two components. Given that the two constituent polymers are semicrystalline, these results demonstrate that each component continues to crystallize in its same native habit following compatibilization by UDCs. Thus, at a temperature (130° C.) above the Tₘ of LDPE, only the P3HB bands are retained. Meanwhile, subtraction of the 130° C. signals from that at room temperature revealed essentially only the LDPE peaks. Also, the P3HB bands disappeared at 180° C., above the Tₘ of P3HB, leaving only a broad amorphous halo. The corresponding SAXS peaks showed qualitatively similar features again illustrating that, even though the blends are compatibilized, there is no mixing at the molecular (or even at the domain) level. The spectra observed by subtracting the 130° C. data from the room temperature data showed that the SAXS LDPE peak moves from a wavevector of q˜0.3 nm⁻¹ (native LDPE) to 0.4 nm⁻¹ in the UDC blend, which implies a reduction in the LDPE long period from ˜21 nm to 16 nm upon compatibilization. This change could be caused by the increased defect content (by crosslinking), a reduction of the equilibrium Tₘ, or an increase in T_{g} due to the P3HB.

TABLE 6
Average diameter (micrometers) for droplets imaged within the
cross-sections of blends both of virgin quality and once crosslinked
with UDCs 1-3 as described (10 micrometers gold, 3 kV).

| Blend | Average Diameter (μm) |
|---|---|
| LDPE-PLLA | 6.6 ± 4.8 |
| PE-PLLA UDC1 | — |
| PE-PLLA UDC2 | — |
| PE-PLLA UDC3 | — |
| Post-Consumer LDPE-PLLA | 17.5 ± 26.5 |
| Post-Consumer LDPE-PLLA UDC3 | — |
| LDPE-P3HB | 5.1 ± 2.6 |
| PE-P3HB UDC1 | — |
| PE-P3HB UDC2 | — |
| PE-P3HB UDC3 | — |
| LDPE-iPP | 3.5 ± 5.4 |
| PE-iPP UDC3 | — |
| PS-PLLA | 18.3 ± 20.4 |
| PS-PLLA UDC3 | — |
| LDPE-iPP-PLLA | 10.3 ± 8.3 |
| LDPE-iPP-PLLA UDC3 | — |

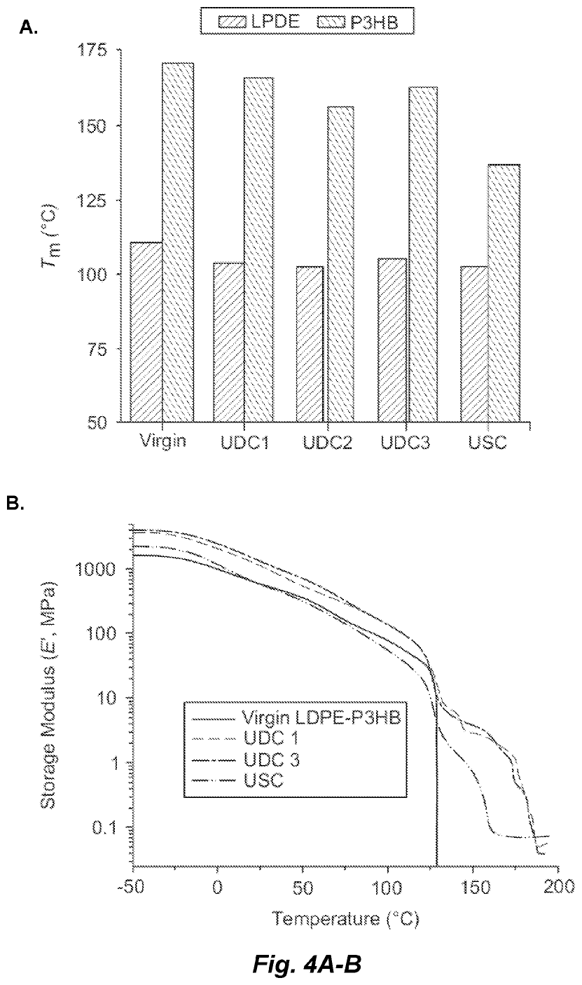

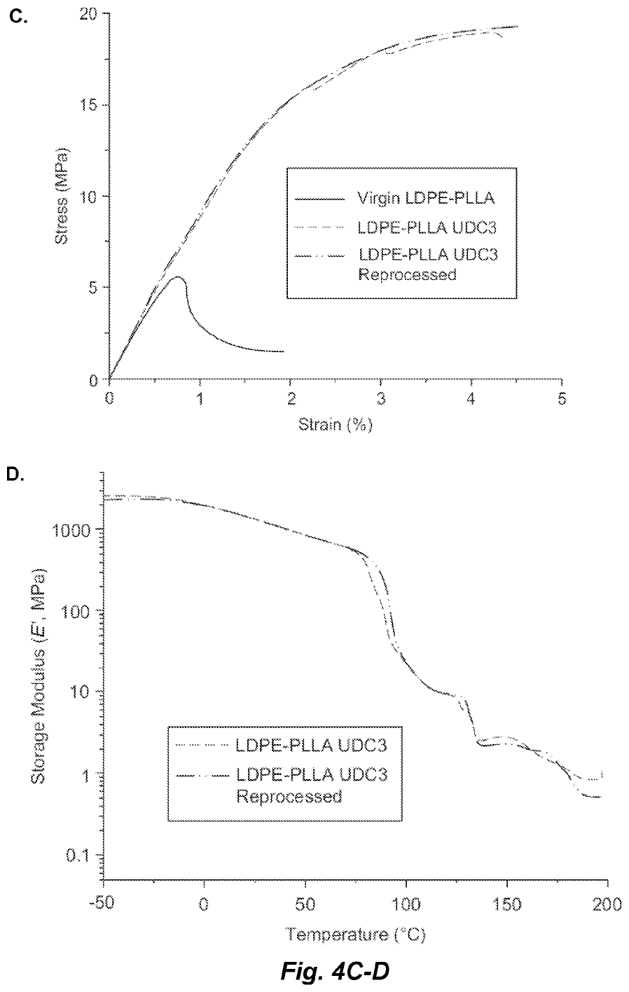

DSC data (FIG. 4a) clearly show that the Tₘ of both constituents in the compatibilized blends is depressed relative to the pure polymer. The Flory theory of polymer crystallization implies that the Tₘ can be depressed by the “copolymer effect”:

$$- \frac{1}{{T}_{m}^{copoly}} - \frac{1}{{T}_{m 0}} = - \frac{R}{{h}_{u}} {X}_{b} ,$$

where Tₘ^{copoly} is the Tₘ of the (graft) copolymer created by the UDC, Tₘ₀ is the Tₘ of the corresponding pure polymer, R is the gas constant, hu is the enthalpy of melting and X_{b} is the crosslinked mole fraction. In the LDPE-P3HB blend the Tₘ of the pure LDPE is 110° C., while that for the LDPE in the UDC systems is ˜104±1° C.; similarly, the pure P3HB has Tₘ=171° C., while the UDC value is 162±4° C. Both of these numbers, which have inherently high uncertainties since they were averaged across the different systems, are consistent with the Flory theory with an X_{b}≈0.02; this is in reasonable accord with the modulus estimates for the pure HDPE systems discussed above. Thus, the experimentally deduced Tₘ values support the formation of the gMBCP.

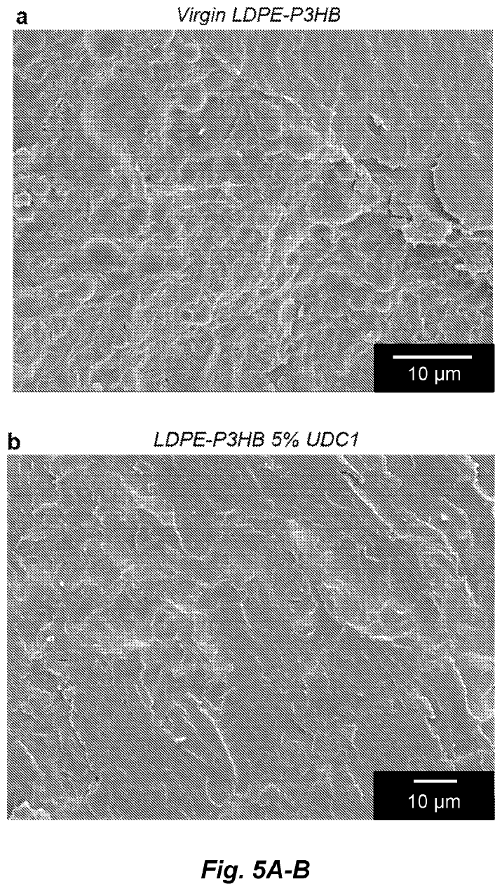

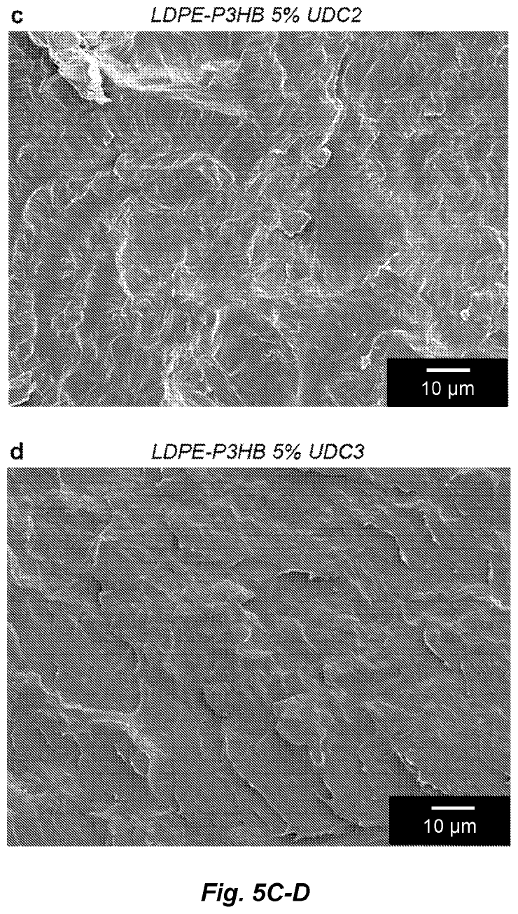

Examining the consequences of these crosslinks on the thermomechanical response revealed that the low-temperature modulus of the UDC-compatibilized blends is larger than the non-compatibilized mixtures (FIG. 4b). This increased modulus evidently is due to the increased connectivity between the phases through crosslinking which permits for better load transfer. In both UDC-compatibilized blends there are two drops in moduli with increasing temperature, corresponding to the melting transitions of the two polymers; however, even at the highest temperature the UDC blends have a residual modulus, because the thermoset character imparted by the UDCs is manifesting even in the melt state. This is similar to the high temperature plateau modulus in the HDPE-UDC system (FIG. 1b). Note that the incompatible blends, mechanically inferior due to the high interfacial tension signaled by large droplets (FIG. 5, Table 5 and 6), fail at the Tₘ of the lower Tₘ polymer. The USC blends behaved in a similar fashion, albeit with notable lower storage modulus than UDC-compatibilized blends across the experimental temperature gradient (FIG. 4b, Table 7).

TABLE 7
Tabulated maximum storage modulus values (−50°
C.) for LDPE-PLA/P3HB blends at virgin and with UDC or
USC incorporation (0.3% strain, 1 Hz, −50-200° C., 5° C./min).

| LDPE-P3HB Blend | E′ₘₐₓ (−50° C., MPa) |
|---|---|
| 0% | 1635 ± 14 |
| 5% UDC1 | 3535 ± 45 |
| 5% UDC3 | 2883.9 ± 11.2 |
| 5% USC | 2179 ± 856 |
| LDPE-PLA Blend | E′ₘₐₓ (−50° C., MPa) |
| 0% | 2795 ± 91 |
| 5% UDC1 | 3714 ± 148 |
| 5% UDC3 | 3008 ± 79 |
| 5% USC | 3634 ± 187 |

| Extruded LDPE-PLA Blend | E′ₘₐₓ (−50° C., MPa) |
|---|---|
| 1^{st} Cycle | 2458 ± 229 |
| Reprocessed 2^{nd} Cycle | 2312 ± 68 |

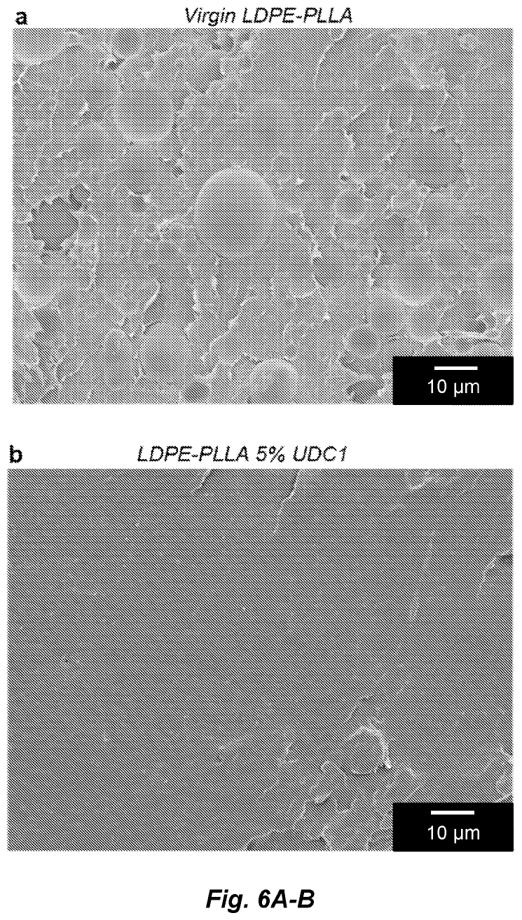

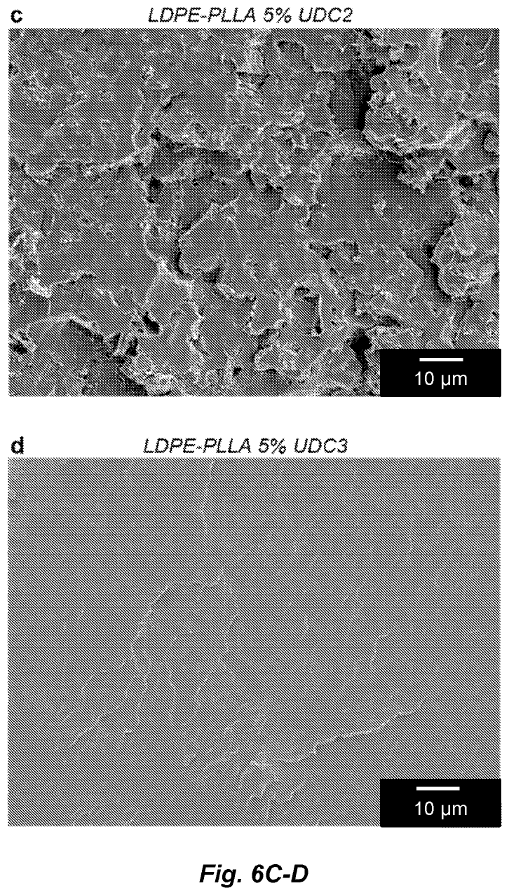

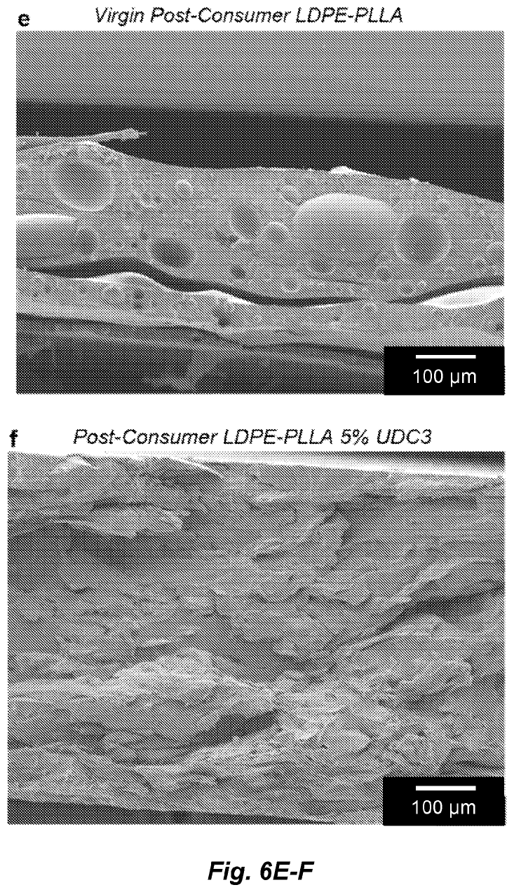

We next examined the reprocessability and recyclability of one canonical example, the UDC-compatibilized LDPE-PLLA blend. UDC3 was selected for this and subsequent studies because UDC3-crosslinked materials showed the best thermomechanical performance, likely due to the stability of the anhydride dynamic linker. UDC3 is also more convenient to operate on than UDC1, as the former does not need an external catalyst to trigger the dynamic exchange. Upon melt-extrusion by a twin-screw extruder, the virgin blend was found to be macro-phase-separated into two individual parts, while the blend embedded with UDC3 was compounded into continuously homogeneous ribbon-like extrudates. The reprocessed specimens are also homogeneous, and the compatibilization was confirmed by the absence of droplets in the cross-section imaging, in contrast to the virgin blend (FIG. 6, Table 5 and 6). The UDC-compatibilized blend is also recyclable via both tensile testing and DMA analyses (FIG. 4c, 4d); SAXS data also support these conclusions. Noteworthy is that the UDC blend showed an enhancement of tensile strength, ultimate strain, and toughness by 346%, 268%, and 1850%, respectively, relative to the virgin blend (Table 7, Table 8). Furthermore, as testament of the “living” nature of the system, addition of 50% virgin LDPE to the post-tensile specimens of the 50:50 LDPE-PLLA blend compatibilized by UDC3 gave a 75:25 LDPE:PLLA blend (2.5% UDC3), which can still be extruded into a homogeneous, reprocessable blend with an enhanced ductility by 220% over the 50:50 blend.

TABLE 8
Tabulated maximum storage modulus values (−50°
C.) for blends of LDPE-PLA/P3HB at virgin and with
UDC/USC incorporation by extrusion (0.3%, 1 Hz, −50°
C.-200° C., 5° C./min).

| | Stress | Strain | Modulus | Toughness |
|---|---|---|---|---|
| Polymer | (MPa) | (%) | (MPa) | (MJ m⁻³) |
| LDPE-PLA | 5.6 ± 0.7 | 1.9 ± 0.8 | 1100 ± 87 | 0.04 ± 0.2 |
| LDPE-PLA 5% UDC3 | 17 ± 2.6 | 3.0 ± 1.0 | 978 ± 18 | 0.34 ± 0.2 |
| LDPE-PLA 5% UDC3 | 19.4 ± 0.6 | 5.1 ± 0.5 | 1003 ± 34 | 0.74 ± 0.1 |
| R1 | | | | |

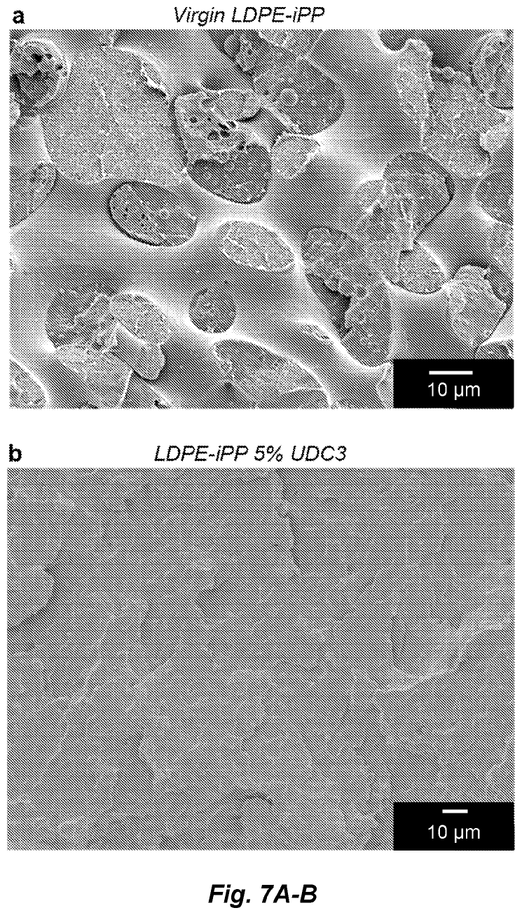

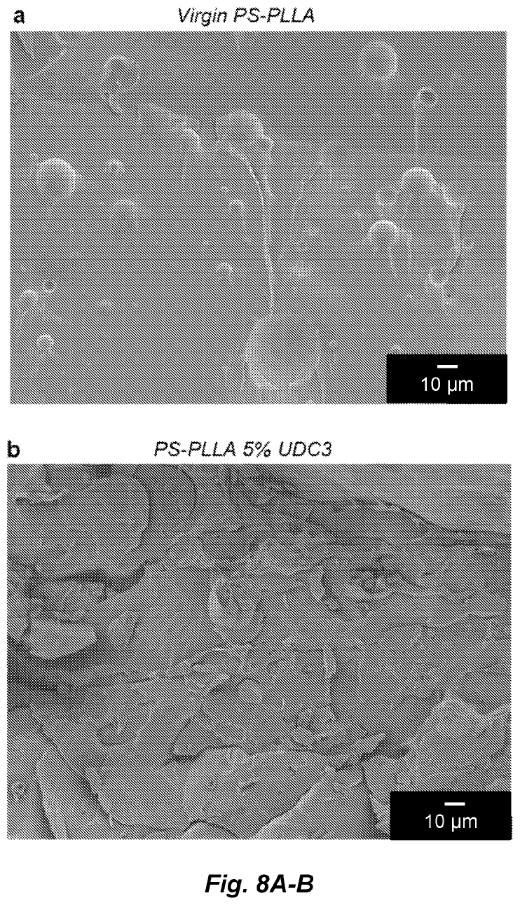

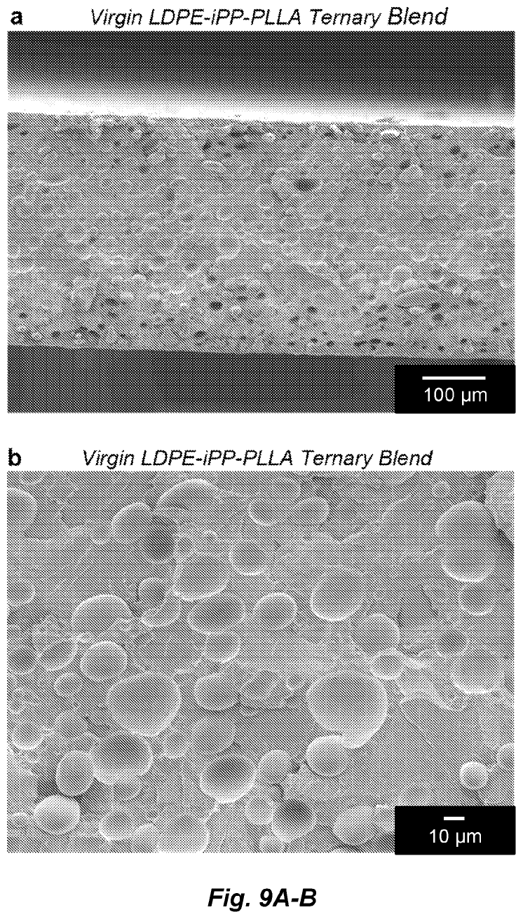

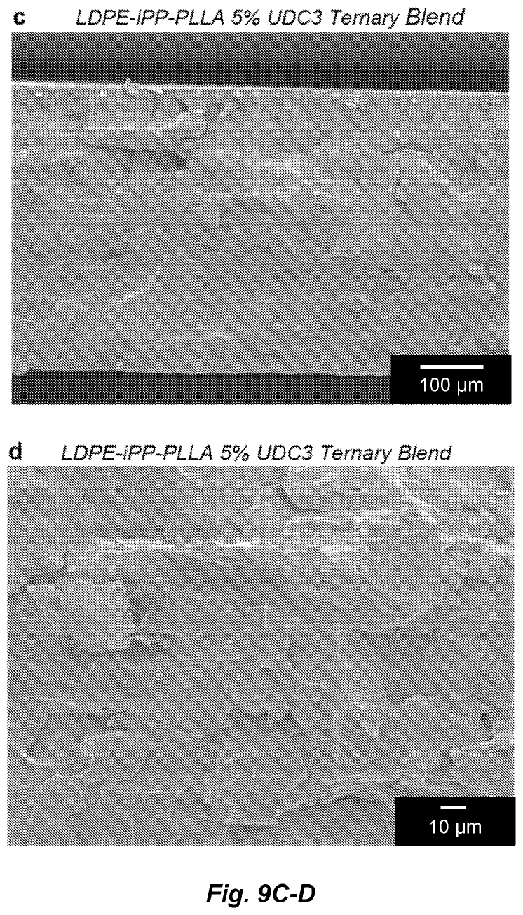

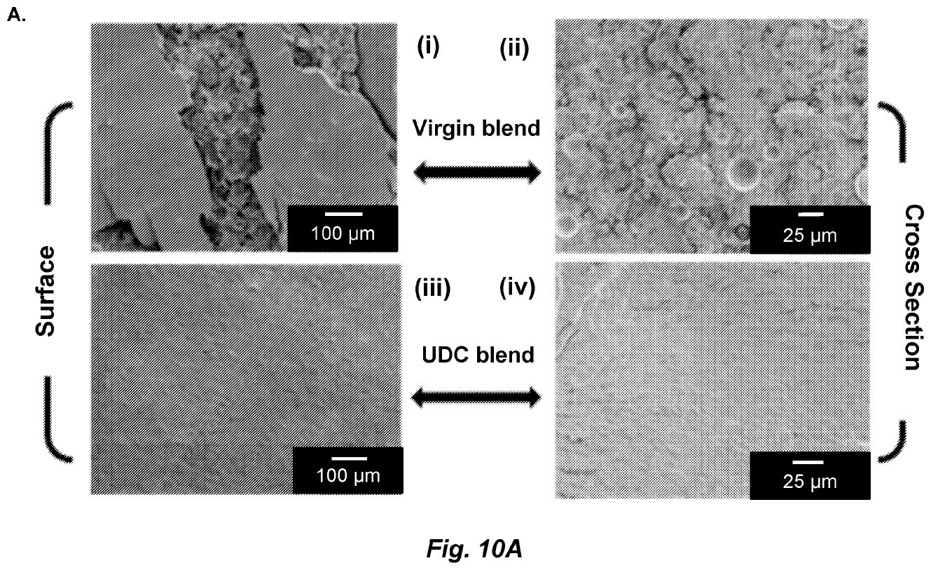

To test the generality of the UDC compatibilization, we also examined other incompatible binary blends and even a more complex ternary blend. Thus, the incompatible LDPE-iPP and polystyrene (PS)-PLLA blends can be readily compatibilized through simple reactive melt-extrusion with UDC3 (FIG. 7, FIG. 8, Table 5 and 6). Remarkably, the UDC compatibilization can go beyond the binary blends. For example, a high degree of compatibilization of the incompatible ternary LDPE-iPP-PLLA blend was successfully achieved by simple melt-extrusion in the presence of 5% UDC3 (FIG. 9, Table 5 and 6). Lastly, to test the tolerance and efficacy of the UDC compatibilization towards dyes or other additives present in the real-world mixed-plastics, mechanically shredded flakes of a LDPE bag and a PLLA cup containing colorants and additives were melt-extruded with 5% UDC3, still achieving successful compatibilization (FIG. 6e, 6f, 11a and Table 5 and 6).

Conclusion. Overall, we credit the enhanced properties, blend compatibilization, and reprocessability of a range of mixed plastics enabled by UDCs to the synergistic combinations of a) inter-chain crosslinks producing gMBCPs through covalently restrained proximity and b) dynamic bond exchange promoting mixing (and, potentially, network relaxation for large enough UDC concentrations). The inter-chain crosslinking generates rapidly evolving, effectively “living” gMBCPs of otherwise immiscible components in situ during processing conditions. This strategy, while relying on the principles of BCP compatibilization, is a molecular solution to circumvent the need for ex-situ synthesized BCP compatibilizers. Linear or grafted MBCPs can achieve blend stabilization by reducing the interfacial tension between incompatible phases. In the scenario of a dynamically crosslinked blend, leveraging rapid crosslink shuffling leads to matrix miscibility due to lowered interfacial tensions between the two domains; this is herein attributed to the formation of “living” gMBCPs. The capacity for these dynamic tethers to flow during processing (mixing) conditions would then be key to achieve balanced inter-chain linking and thus stabilization of the binary blend. From these observations we conclude that both melt-processability and inter-chain BCP architectures, uniquely accessible by UDCs, are crucial to achieving compatibility between two polar extremes and thus highlight the broader utility of dynamic crosslinking. The universal aspect of UDC carbene C—H insertion and dynamic-crosslinking-induced compatibilization principles introduced here are applicable across binary, ternary, and “post-consumer” blends studied herein. This UDC compatibilization strategy may thus prove to be a facile means to achieve the ultimate goal of reusing mixed plastic waste over multiple use cycles.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

### Materials.

Reagents 4-bromobenzenethiol, pentaerythritol tetrakis(3-mercaptopropionate) (PETMP), anhydrous ammonia (lecture bottle, 170 g), and 4-(trifluoroacetyl)benzoic acid were purchased from Sigma-Aldrich. TDBA {4-[3-(Trifluoromethyl)-3H-diazirin-3-yl]benzoic acid} and DBU {1,8-diazabicyclo[5.4.0]-7-undecene} were purchased from TCI America. All reagents were used as received. Poly(methyl methacrylate) (PMMA, powder, Mₙ=120 kg mol⁻¹, 182230), isotactic polypropylene (iPP, 5 mm gran., Mₙ=97.0 kg mol⁻¹, 427861), low-molecular-weight polyethylene (LMWPE, powder, Mₙ=1.7 kg mol⁻¹, 42772), poly-3-hydroxybutyrate (P3HB, powder, natural origin, 363502), and polystyrene (PS, 2-4 mm gran., M_{w}=35,000, 1002546287) were purchased from Sigma-Aldrich. Low-density polyethylene (LDPE, 5 mm gran., MFI=21, ET31-GL-000105), high-density polyethylene (HDPE, 2-4 mm gran., MFI=7.6, ET32-GL-000110), and poly(L-lactic acid) (PLLA, 3-5 mm gran., MFI=8, ME34-GL-000120) were purchased from Goodfellow. Ultra-high molecular weight polyethylene (UHMWPE, powder, 43951-30) was purchased from Alfa Aesar. All polymers were used as received.

### Instruments and Characterizations.

Nuclear magnetic resonance (NMR) data were recorded on a Bruker 400 MHz or 500 MHz spectrometer at 298 K. Chemical shifts (δ) are reported in ppm with the solvent resonance employed as the internal standard (chloroform at 7.26 ppm for ¹H-NMR and 77.16 ppm for ¹³C NMR spectroscopy; acetone-d₆ at 2.05 ppm for ¹H NMR and 29.8 for ¹³C NMR spectroscopy). Signals are reported as integration, multiplicity (s=singlet, d=doublet, t=triplet, q=quartet, m=multiplet or unresolved, br=broad signal), coupling constant(s) in Hz, assignment. For diastereomeric mixtures only signals belonging to the major diastereomer are reported, unless stated otherwise.

High resolution mass spectrometric (HRMS) analyses were performed at the Mass Spectrometry Core Facility at Columbia University. Measurements were conducted on a Waters high-resolution Xevo G2-XS Q-TOF mass spectrometer coupled with an acquity UPLC H-Class in positive electrospray ionization mode (ESI). Infrared spectra (IR) were measured neat as thin films or solids on a Perkin Elmer Spectrum Two FT-IR spectrometer. Only major peaks are reported as absorption maxima (n, cm-1).

Super critical fluid chromatography (SFC) was carried out on a Waters low resolution SQD2 quadrupole mass spectrometer equipped with a UPC2 SFC inlet, a photodiode array UV-vis detector, and a dual ESI/APCI probe in positive ionization mode at the Mass Spectrometry Core Facility at the Columbia University chemistry department. Separation was performed on a Waters BEH (1.7 um, 2.1×100 mm) analytical column using CO₂/acetonitrile gradient elution.

Thermogravimetric Analysis (TGA) was performed on UDCs and dried polymer samples (5-10 mg) on a Q50 TGA Analyzer (TA Instruments). Polymer samples were placed in a weight-tared aluminum pan and heated from 25° C. to 700° C. at a rate of 10° C./min. UDCs were similarly, individually prepared and heated from 25° C. to 200° C. A constant flow of N₂ was flushed through the furnace at 40.00 mL/min, and the mass loss was quantified as a percentage of the initial mass with increased temperature. Thermal stability and weight loss measurements were analyzed on Universal Analysis (TA).

Differential Scanning calorimetry (DSC) was performed on dried samples (3-5 mg) on an Auto Q20 in aluminum pans and hermetic lids against an identical, empty reference pan/lid (TA Instruments). The oven was constantly flushed with 40.00 mL/min of N₂. UDC sample analysis (0 to 200° C.) was performed on the 1^{st} heating scan, where single-event exothermic signals for carbene formation were recorded at a heating rate of 5° C./min. On the other hand, DSC plots for polymer species show the 2^{nd} heating scan and 1^{st} cooling scan (−40 or −50 to 200° C.). Initial (1^{st}) heating scans were operated at a ramp rate of 10° C./min, followed by cooling at 10° C./min and a 2^{nd} heating scan at 10° C./min. Thermal transitions by DSC were characterized by Universal Analysis (TA). Explosivity and shock sensitivity values for UDC1, 2, and 3 were obtained by DSC measurements.

Fourier Transform Infrared Spectroscopy (FTIR) experiments were carried out on a Bruker TENSOR II FT-IR spectrometer fitted with a Platinum ATR between 4000-400 cm-1. FTIR data was acquired and analyzed through the software OPUS (Bruker).

SAXS/WAXS experiments were performed on dried samples using a lab-scale instrument (SAXSlab, Amherst, MA) with a beam cross section of 200×200 μm and wavelength λ=0.154 nm for variable sample-to-detector distances covering a q range of 0.05-12 nm⁻¹. Samples were ˜1 mm thick and cut down to ˜0.25 cm² in area. The data were background subtracted and reported as scattered intensity as a function of momentum transfer, q. 9 spots were rastered across each sample to provide averaged data. Temperature control was provided via a Linkam stage (Linkam Scientific, Redhill, UK). Temperature protocol involved taking data at 30° C. for reference, heating at 10° C./min up to 120° C., then 130° C. and 180° C. prior to cooling back to 30° C. for sample recrystallization. Samples were held at each temperature for 1.5 h with data collected in 5-min exposures to track progression at each temperature. Data presented in FIG. 4 represents the final exposure at each temperature.

### Preparation, Processing, and Characterization of Blends and Films.

Method 1 (general method): commercial polymer pellets were dissolved in a compatible solvent (toluene for PE and PS, CHCl₃ for PLLA and P3HB, C₂Cl₄ for iPP) with an oil bath (80° C.) when necessary. Mixtures were combined into a 500 mL round bottom flask and stirred until suitably mixed. After ensuring the temperature of the solution was below 85° C., the crosslinker was solubilized in acetone and added to the mixed solution. The round bottom flask containing the blended polymers and crosslinker was then placed in an ice bath and cooled. Next, the solvent was removed under vacuum, and the residual polymer blend was extensively dried in a vacuum oven at 50° C. until no weight change was noted.
Method 2 (LDPE-iPP): iPP (2.5 g) and LDPE (2.5 g) were solubilized, precipitated, and then ground into a powder with a mortar and pestle. A selected UDC (250 mg, 5% by weight) was added to the dry blend and physically mixed into a mixture again with the mortar and pestle.
Method 3 (“post-consumer” bag and cup): Flakes from a PLLA cup and a LDPE bag (2.5 g each) were placed in a glass vial. A selected UDC (250 mg, 5%) was dissolved in 20 mL acetone and added to the vial, which was then sealed and placed in a light-free environment for ˜6 h to allow for swelling of the materials. Acetone was removed under vacuum, and the residue blend was extensively dried in a vacuum oven at 50° C. until no weight change was noted.

Melt extrusion processing and reactive blending of the blends were conducted using a Thermo Fisher Scientific HAAKE™ Minilab 3 Micro-Compounder (Twin Screw Extruder) in the manual mode. The compounder was operated at 180° C. with inert gas (Ar) flowing through the chamber. The screws were set to 50 rpm with care that the torque was not above 5 Nm. The material was then added through a hopper and pushed down to the barrel. The compounder was set to flush mode, and material was directly extruded through the standard flat filament die over 10-15 min. The extruded material was then compression molded for mechanical testing or directly taken for SEM imaging.

Film processing (melt compression molding) was performed for all polymers using a Carver Auto Series Plus Laboratory Press (Carver, Model 3889.1PL1000, Max Force 15 ton) with programmable electrically heated platen (EHP) temperature, and air-/water-controlled cooling. Polymer samples prepared for film processing (3-5 g) were sandwiched between thin, non-stick Teflon sheets in a stainless-steel mold with inset dimensions 30×73.5×0.87 mm fabricated in-house and compressed between two 6″×6″ EHPs at a set pressure of 7000 psi for 30-60 min. LMWPE and LDPE samples were heated to 110° C. HDPE, UHMWPE, and PMMA samples were heated to 150° C. Blends containing PLLA or P3HB were heated at 175° C. All samples were cooled in ˜10 minutes by air- and water-circulation attachments while under the 7 kpsi of compression.

Tensile stress/strain tests were performed on ASTM D-638 Standard Type-V tensile bar (“dog-bones”; w=3.18 mm; t=0.5-1.0 mm) specimens on an Instron 5966 (TA Instruments) universal testing system equipped with a 10 kN load cell and operated at room temperature. Selected samples were tightly placed between textured grips, with a grip separation of ˜28 mm, and pulled at a constant strain-rate of 5 mm/min until break. Reported curves and values were averaged from 3-4 individual trials of each sample for reproducibility, and error margins are supplied accordingly. Force/displacement values were recorded and normalized to stress/strain by the Bluehill Universal Software (TA). Young's modulus (E, MPa), ultimate strength (OB, MPa), yield strength (MPa), and elongation at break (&B, %) were obtained from the software analysis. Toughness values (Ur; MJ m 3) were obtained by manual calculation (integration) of the area under the stress/strain curve.

Dynamic mechanical analysis (DMA) was performed on a Q800 DMA Analyzer (TA Instruments). Samples for DMA were prepared under similar conditions (w=5.40 mm, t=0.5-1.0 mm) with length measured within the grips by the Q-series measurement software (TA) before each trial. Grips were judiciously tightened between 3-7 lbs—in accordance with the sample's modulus. Temperatures below ambient were probed by a liquid-nitrogen GCA tank attachment (TA).

Bulk flow experiments by oscillatory shear rheology were performed on a Discovery Series Hybrid 2 (DHR-2) Rheometer (TA Instruments) between 130-160° C. in step-transient creep-recovery testing mode. Circular discs (25 mm) of polymer films were loaded between two 25 mm steel parallel-plate EHP geometries under N₂ (30 psi) gas flow and allowed to soak at specified temperatures for a minimum of 5 min. Rheometer control and data analysis were performed with the TA Instruments TRIOS Software.

Scanning electron microscopy (SEM) was employed for surface analysis and imaging of UDC-treated and non-treated polymer blends and carried out on a JEOL JSM-6500F scanning electron microscope at an electron voltage of 3 kV, as described in image captioning. Electron current was set to 7, and the working distance was set to 10 mm. Samples were coated with a thin layer (10 nm, 33 seconds) of gold using a plasma sputter-coater under argon (Desk II). Coated films were inserted into the microscope on an aluminum mount, secured with double-sided carbon tape and copper tape. Image processing was conducted using the publicly available tool ImageJ2 (NIH).

### Example 2. Synthetic Methods

All reactions were performed in flame-dried glassware under an atmosphere of N₂, unless stated otherwise. Reactions were stirred magnetically and monitored by analytical thin layer chromatography (TLC). TLC was performed on Merck silica gel 60 F₂₅₄ TLC glass plates and visualized with 254 nm light and aqueous potassium permanganate followed by heating. Crude products were absorbed into silica and purified by flash column chromatography on SiliaFlash, SiliCycle Silica Gel P60, 230-400 mesh, 40-63 μm particle size and analytical grade hexane, ethyl acetate, dichloromethane, and methanol as eluents at 0.3-0.5 bar overpressure. Preparative thin-layer chromatography (PTLC) was carried out on SiliCycle 1000 μm 20×20 cm TLC plates and visualized with a low intensity 365 nm UV-lamp Concentration under reduced pressure (in vacuo) was performed by rotary evaporation at 40° C. at the appropriate pressure unless otherwise noted. The yields given refer to the purified products, unless otherwise stated. For larger scale synthesis, 4-[3-(trifluoromethyl)-3H-diazirin-3-yl]benzoic acid (TDBA) was prepared following a known literature procedure (Asian. J. Org. Chem. 2015, 4, 724).

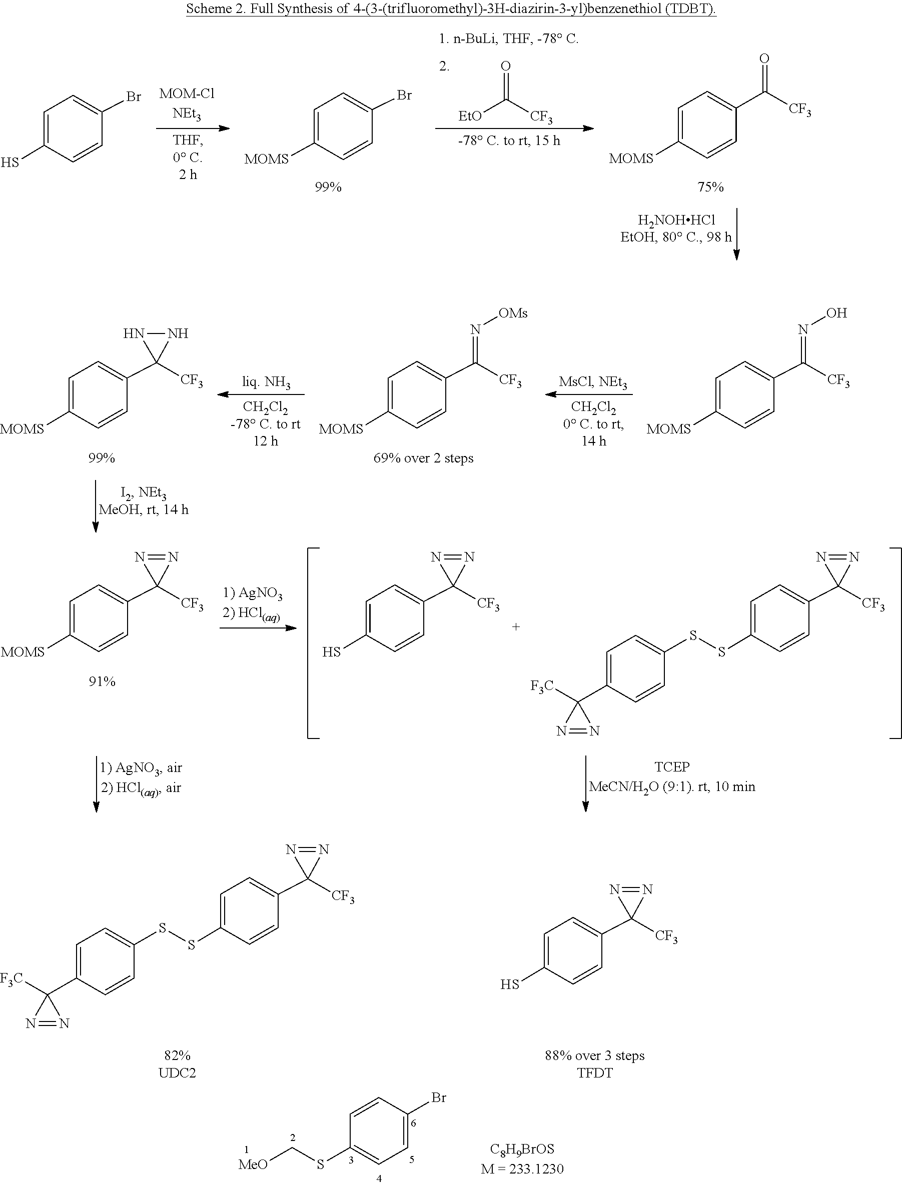

### Synthesis of (4-bromophenyl)(methoxymethyl)sulfane

To a solution of 4-bromothiophenol (4.0 g, 21.2 mmol, 1.0 equiv.) in anhydrous THF (40 mL) was added triethylamine (5.92 mL, 42.4 mmol, 2.0 equiv.), and the resulting solution cooled to 0° C. by means of an ice bath. Chloromethyl methyl ether (2.4 mL, 31.8 mmol, 1.5 equiv.) was added dropwise under vigorous stirring at 0° C., resulting in a sticky voluminous precipitate. The reaction mixture was stirred for 2 h at 0° C., after which the mixture was diluted with water and the aqueous phase extracted with ethyl acetate (3×). The combined organic phases were dried (MgSO₄), filtered and concentrated in vacuo. The resulting crude mixture was purified by silica flash column chromatography (eluent: 2% ethyl acetate in hexanes), yielding the title product (4.79 g, 20.5 mmol, 97%) as colorless oil. R_{f} (20% ethyl acetate in hexanes): 0.58, ¹H NMR (500 MHZ, CDCl₃) δ: 7.41 (2H, d, J=8.6 Hz, H₄), 7.33 (2H, d, J=8.6 Hz, H₅), 4.93 (2H, s, H₂), 3.43 (3H, s, H₁); ¹³C NMR (126 MHZ, CDCl₃) δ: 135.3 (C₃), 132.1 (2C, C₄), 131.8 (2C, C₅), 120.9 (C₆), 77.8 (C₂), 56.2 (C₁); m/z HRMS ASAP+ found [M{⁷⁹Br}+H]⁺ 231.9548, found [M{⁸¹Br}+H]⁺ 233.9525, C₈H₁₀⁷⁹BrOS⁺ requires 231.9557, C₈H₁₀⁸¹BrOS⁺ requires 233.9537.

### Improved synthesis of (4-bromophenyl)(methoxymethyl)sulfane

A solution of 4-bromothiophenol (0.527 g, 2.65 mmol, 1.0 equiv.) and triethylamine (0.74 mL, 5.3 mmol, 2.0 equiv.) in THF (5.0 mL) was cooled to 0° C. Chloromethyl methyl ether (2.92 mmol, 1.1 equiv.) in THF (1.0 mL) was added resulting in the formation of a white precipitate. The suspension was stirred at 0° C. for 2 h. Additional THF (5.0 mL) was added to dilute the viscous mixture and the reaction was stirred at room temperature for 2 h. The reaction was diluted with water and extracted with EtOAc (3 times). The organic phases were combined, dried over MgSO₄, filtered, and concentrated. The crude mixture was analyzed by ¹H NMR spectroscopy using 1,3,5-trimethoxybenzene as an internal standard. The yield of (4-bromophenyl)(methoxymethyl)sulfane was determined to be 94% (2.49 mmol). Note: technical-grade chloromethyl methyl ether was used for this experiment. The purity of the reagent was determined by ¹H NMR spectroscopy with 1,3,5-trimethoxybenzene as internal standard prior to use.

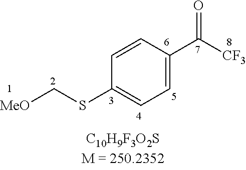 [SMILES: COCSc1ccc(C(=O)C(F)(F)F)cc1]

### Synthesis of 2,2,2-trifluoro-1-(4-((methoxymethyl)thio)phenyl)ethan-1-one

A solution of (4-bromophenyl)(methoxymethyl)sulfane (4.79 g, 20.5 mmol, 1.0 equiv) in anhydrous THF (100 mL) was cooled to −78° C. A solution of n-BuLi (2.5 m in hexanes, 9.48 mL, 24.6 mmol, 1.2 equiv) was added dropwise with stirring and the resulting solution was stirred for 1 h at −78° C. Ethyl trifluoroacetate (2.93 mL, 24.6 mmol, 1.2 equiv) was then added dropwise at −78° C. After complete addition, the cooling was removed, and the reaction mixture stirred for 14 h at room temperature. The mixture was quenched by addition of saturated ammonium chloride solution and the resulting biphasic mixture was extracted with ethyl acetate (3×). The combined organic phases were dried (MgSO₄), filtered and concentrated in vacuo. The resulting crude oil was purified by silica flash column chromatography (eluent: 3% ethyl acetate in hexanes), yielding the title product (3.40 g, 13.6 mmol, 66%) as a colorless oil. R/(15% ethyl acetate in hexanes): 0.46 (diffuse); ¹H NMR (500 MHZ, CDCl₃) δ: 7.96 (2H, d, J=8.8 Hz, H₄), 7.56 (2H, d, J=8.8 Hz, H₅), 5.09 (2H, s, H₂), 3.46 (3H, s, H₁); ¹³C NMR (126 MHz, CDCl₃) δ: 179.8 (q, 2J=34.8 Hz, C₇), 147.7 (C₃), 130.5 (2C, C₄), 127.6 (2C, C₅), 127.2 (C₆), 116.9 (q, ¹J=291.3 Hz, C₈), 76.7 (C₂), 56.5 (C₁); ¹⁹F NMR (471 MHZ, CDCl₃) δ: −70.4; m/z HRMS ASAP+ found [M+H]⁺ 251.0359, C₁₀H₁₀F₃O₂S⁺ requires 251.0354.

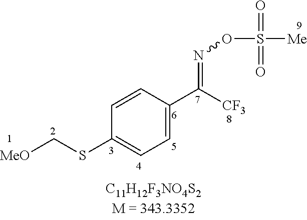 [SMILES: COCSc1ccc(C(=NOS(C)(=O)=O)C(F)(F)F)cc1]

### Synthesis of 2,2,2-trifluoro-1-(4-((methoxymethyl)thio)phenyl)ethan-1-one O-methyl-sulfonyl oxime

### Step 1: Oxime Formation.

2,2,2-trifluoro-1-(4-((methoxymethyl)thio)phenyl)ethan-1-one (3.40 g, 13.6 mmol, 1.0 equiv), hydroxylamine hydrochloride (4.25 g, 61.2 mmol, 4.5 equiv) and sodium hydroxide (2.44 g, 61.2 mmol, 4.5 equiv) were dissolved in anhydrous ethanol (50 mL). The vessel was sealed under nitrogen and the mixture stirred vigorously at 80° C. for 18 h. The suspension was allowed to cool to room temperature and subsequently diluted with ethyl acetate, washed with dilute aqueous hydrochloric acid (0.1 M, 3×) and brine. The organic phase was then dried (MgSO₄), filtered, and concentrated in vacuo. The crude oxime was obtained as a clear oil and used directly in the next step.

### Step 2: Mesylation.

The crude oxime obtained from step 1 was dissolved in anhydrous dichloromethane (40 mL) and the resulting solution cooled to 0° C. by means of an ice-bath. Triethylamine (3.03 mL, 21.8 mmol, 1.6 equiv) was added dropwise to the stirred solution at 0° C., followed by dropwise addition of methanesulfonyl chloride (1.05 mL, 13.6 mmol, 1.0 equiv). The cooling was then removed, and the reaction mixture stirred at room temperature for 14 hours. Equal parts of water and saturated aqueous ammonium chloride solution were then added, and the resulting biphasic mixture extracted with diethyl ether (3×). The combined organic phases were dried (MgSO₄), filtered, and concentrated in vacuo. The residue was purified by silica flash column chromatography (eluent: 100% dichloromethane), yielding the title product (3.97 g, 11.6 mmol, 85%, d.r.=1.5:1) as a pale yellow oil that solidifies on standing. R_{f} (100% dichloromethane): 0.64; ¹H NMR (400 MHz, CDCl₃) δ: 7.58-7.48 (3H, m, H_{4,5}), 7.45-7.37 (1H, m, H_{4,5}), 5.044 (2H, s, H₂, major) [5.037 (2H, s, H₂, minor)], 3.45 (3H, s, H₁, major) [3.44 (3H, s, H₁, minor)], 3.25 (3H, s, H₉, major) [3.24 (3H, s, H₉, minor]; ¹³C NMR (101 MHz, CDCl₃) δ: 154.5 (q, ³J_{F}=33.7 Hz, C₇, major) [154.6 (q, ²J_{F}=32.6 Hz, C₇, minor)], 142.65 (C₃, major) [142.74 (C₃, minor)], 129.1 (C_{4/5}, major) [129.4 (C_{4/5}, minor)], 128.4 (C_{4/5}, major) [128.7 (C_{4/5}, minor)], 121.6 (C₆, major) [124.9 (minor)], 119.7 (q, ¹J_{F}=278.2 Hz, C₈, major), [117.4 (q, ¹J_{F}=283.6 Hz, C₈, minor)], 76.3 (C₂, major) [76.4 (C₂, minor)], 56.3 (C₁), 36.8 (C₉, major) [36.9 (C₉, minor)]; ¹⁹F NMR (376 MHZ, CDCl₃) δ: −66.21 (major) [−61.4 (minor)]. m/z HRMS ESI+ found [M+H]⁺ 344.0242, C₁₁H₁₃F₃NO₄S₂⁺ requires 344.0238.

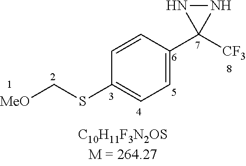 [SMILES: COCSc1ccc(C2(C(F)(F)F)NN2)cc1]

### Synthesis of 3-(4-((methoxymethyl)thio)phenyl)-3-(trifluoromethyl)diaziridine

Anhydrous ammonia was condensed (approx. 15 mL) into a pre-cooled flask at −78° C. using a dry-ice acetone cold finger. To the stirred liquid ammonia, a solution of 2,2,2-trifluoro-1-(4-((methoxymethyl)thio)phenyl)ethan-1-one O-methylsulfonyl oxime (3.79 g, 11.6 mmol, 1.0 equiv) in anhydrous dichloromethane (9 mL) was added dropwise at −78° C. The solution was stirred for 1 h at −78° C. and then gradually warmed to room temperature over the course of 12 h, while allowing excess ammonia to vent. To the residue, water (40 mL) was added, and the resulting solution was extracted with dichloromethane (3×20 mL). The combined organic phases were washed with brine, then dried (MgSO₄), filtered and concentrated in vacuo. The resulting crude oil was purified by silica flash column chromatography (gradient elution: 25% dichloromethane in hexanes to 100% dichloromethane), yielding the title product (2.29 g, 8.67 mmol, 75%) as a colorless oil. R_{f} (100% dichloromethane): 0.29; ¹H NMR (500 MHZ, CDCl₃) δ: 7.55-7.47 (4H, m, H_{4,5}), 4.99 (2H, s, H₂), 3.44 (3H, s, H₁), 2.68 (1H, br s, NH), 2.17 (1H, br s, NH); ¹³C NMR (126 MHz, CDCl₃) δ: 142.3 (C₃), 129.8 (C₆), 129.5 (C_{4/5}), 128.7 (C_{4/5}), 123.5 (q, ¹J_{F}=278.3 Hz, C₈), 77.1 (C₂), 57.9 (q, ²J_{F}=36.3 Hz, C₇), 56.2 (C₁). ¹⁹F NMR (471 MHz, CDCl₃) δ: −74.59; m/z HRMS ESI+ found [M+H]⁺ 265.0635, C₁₀H₁₂F₃N₂OS⁺ requires 265.0623.

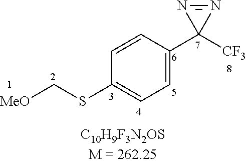 [SMILES: COCSc1ccc(C2(C(F)(F)F)N=N2)cc1]

### Synthesis of 3-(4-((methoxymethyl)thio)phenyl)-3-(trifluoromethyl)-3H-diazirine

To a solution of 3-(4-((methoxymethyl)thio)phenyl)-3-(trifluoromethyl)diaziridine (2.29 g, 8.67 mmol, 1.0 equiv) in methanol (60 mL) was added triethylamine (3.62 mL, 26.0 mmol, 3.0 equiv) followed by portion-wise addition of iodine (6.60 g, 26.0 mmol, 3.0 equiv) with vigorous stirring at room temperature. The resulting dark solution was stirred for 14 h at room temperature under the exclusion of ambient light (tin foil) and subsequently quenched via addition of 10% aqueous sodium thiosulphate solution (60 mL). The almost colorless biphasic mixture was extracted with diethyl ether (3×), and the combined organic phases dried (MgSO₄), filtered, and concentrated under reduced pressure. The residue was purified by silica flash column chromatography (elution: 25% dichloromethane in hexanes), yielding the title product (1.92 g, 7.32 mmol, 84%) as a pale yellow oil. R_{f} (50% dichloromethane in hexanes): 0.62; ¹H NMR (400 MHZ, CDCl₃) δ: 7.48 (2H, d, J=8.7 Hz, H_{4/5}), 7.11 (2H, d, J=8.7 Hz, H_{4/5}), 4.98 (2H, s, H₂), 3.43 (3H, s, H₁); ¹³C NMR (101 MHZ, CDCl₃) δ: 139.0 (C₃), 129.6 (2C, C_{4/5}), 127.2 (C₆), 127.0 (2C, C_{4/5}), 122.2 (q, ¹J_{F}=274.7 Hz, C₈), 77.0 (C₂), 56.2 (C₁), 28.4 (q, ²J_{F}=28.4 Hz, C₇); ¹⁹F NMR (376 MHz, CDCl₃) δ: −65.40.

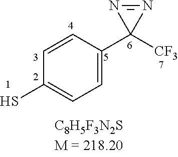 [SMILES: FC(F)(F)C1(c2ccc(S)cc2)N=N1]

### Synthesis of 4-(3-(trifluoromethyl)-3H-diazirin-3-yl)benzenethiol

A solution of 3-(4-((methoxymethyl)thio)phenyl)-3-(trifluoromethyl)-3H-diazirine (4.00 g, 15.3 mmol, 1.0 equiv) in anhydrous ethanol (450 mL, degassed by sparging with N₂ for 20 min) was heated to 55° C. and silver nitrate (3.11 g, 18.3 mmol, 1.2 equiv) was added in one portion with vigorous stirring. The mixture was stirred at 55° C. for 2 h, during which the reaction mixture became considerably more viscous followed by the formation of a voluminous precipitate. At this point silver nitrate (778 mg, 4.58 mmol, 0.25 equiv) was added and stirring was continued at 55° C. for 1 h. All volatiles were removed in vacuo using a rotary evaporator. The reaction flask was evacuated and backfilled with N₂ three times, and 6 M aqueous hydrochloric acid (380 mL, degassed by sparging with N₂ for 20 min) was added via cannula. The resulting suspension was stirred for 30 min before dichloromethane (160 mL, degassed by sparging with N₂ for 20 min) was added via cannula. The mixture was vigorously stirred for 1.0 h at room temperature. The reaction mixture was diluted with dichloromethane, and the organic phase washed with water and brine. The organic phase was dried (MgSO₄), filtered and concentrated in vacuo.

TLC and ¹H-NMR analysis of crude reaction mixture indicated the presence of disulfide UDC2. Hence, the crude mixture was dissolved in acetonitrile (180 mL) and water (20 mL) was added. To the resulting solution tris(2-carboxyethyl) phosphine (TCEP, 1.09 g, 3.83 mmol, 0.25 equiv) was added in one portion. The mixture was stirred at room temperature for 20 min diluted with water (200 mL) and extracted with diethyl ether (3×150 mL). The combined organic phases were dried over sodium sulfate, filtered and concentrated. Purification by silica flash column chromatography (elution: 0.2% diethylether in hexanes) yielded the title product (122 mg, 559 μmol, 73%) as a volatile pale-yellow oil. R_{f} (10% diethylether in hexanes): 0.28; ¹H NMR (400 MHZ, CDCl₃) δ: 7.29 (2H, d, J=8.6 Hz, H₃), 7.08 (2H, d, J=8.6 Hz, H₄), 3.54 (1H, s, H₁); ¹³C NMR (101 MHZ, CDCl₃) δ: 134.2 (C₂), 129.3 (2C, C₄), 127.3 (2C, C₃), 126.4 (C₅), 122.2 (q, ¹J=274.6 Hz, C₇), 28.4 (q, 2J=40.5 Hz, C₆); ¹⁹F NMR (376 MHZ, CDCl₃) δ: −65.4.

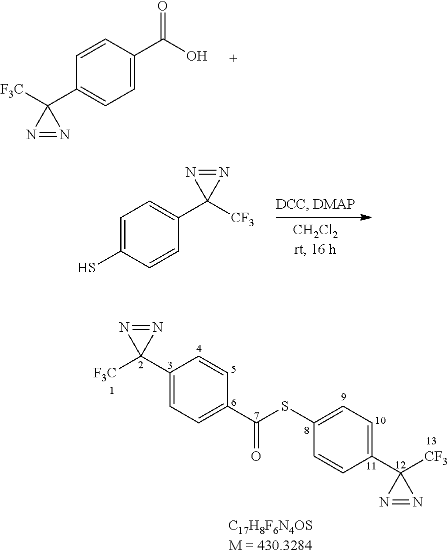 [SMILES: O=C(O)c1ccc(C2(C(F)(F)F)N=N2)cc1 ; FC(F)(F)C1(c2ccc(S)cc2)N=N1 ; O=C(Sc1ccc(C2(C(F)(F)F)N=N2)cc1)c1ccc(C2(C(F)(F)F)N=N2)cc1]

### Synthesis of S-(4-(3-(trifluoromethyl)-3H-diazirin-3-yl)phenyl) 4-(3-(trifluoromethyl)-3H-diazirin-3-yl)benzothioate (UDC1)

A 50 mL Schlenk flask was charged with 4-(3-trifluoromethyl)-3H-diazirin-3-yl)benzoic acid (TDBA) (2.0 g, 8.69 mmol, 1.0 equiv), DCC (2.15 g, 10.4 mmol, 1.2 equiv) and DMAP (425 mg, 3.48 mmol, 0.4 equiv), sealed and backfilled with nitrogen (3×). A solution of 4-(3-(trifluoromethyl)-3H-diazirin-3-yl)benzenethiol (TDBT) (1.90 g, 8.69 mmol, 1.0 equiv) in anhydrous dichloromethane (29 mL) was added via syringe, and the resulting mixture stirred at room temperature for 16 h. The reaction mixture was filtered through celite, the filter cake washed with diethyl ether, and the filtrate concentrated in vacuo. The residue was purified by silica flash column chromatography (gradient elution: hexanes then 8% dichloromethane in hexanes), yielding the title product (3.51 g, 8.16 mmol, 94%) as a colorless waxy solid. R_{f} (10% diethyl ether in hexanes): 0.63; ¹H NMR (400 MHZ, CDCl₃) δ: 8.02 (2H, d, J=8.7 Hz, H₅), 7.55 (2H, d, J=8.7 Hz, H₄), 7.31 (2H, d, J=8.2 Hz, H_{9/10}), 7.28 (2H, d, J=8.2 Hz, H_{9/10}); ¹³C NMR (101 MHZ, CDCl₃) δ: 188.4 (C₇), 137.2 (C₆), 135.3 (2C, C₉), 134.9 (C₃), 130.9 (C_{8/11}), 129.1 (C_{8/11}), 128.0 (2C, C₅), 127.4 (2C, q, 4J=1.5 Hz, C₁₀), 127.0 (2C, q, 4J=1.8 Hz, C₄), 122.1 (q, ¹J=274.9 Hz, C_{1/13}), 121.9 (q, ¹J=274.9 Hz, C_{1/13}), 28.6 (q, 2J=40.9 Hz, C_{2/12}), 28.5 (q, 2J=40.9 Hz, C_{2/12}); ¹⁹F NMR (282 MHZ, CDCl₃) δ: −64.0, −64.2 IR (film) vₘₐₓ: 3198, 2927, 1722, 1699, 1253, 1179, 1128, 905.

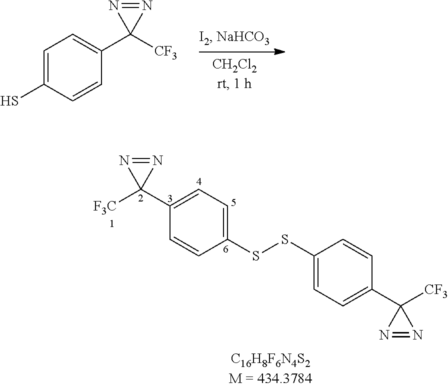 [SMILES: FC(F)(F)C1(c2ccc(S)cc2)N=N1 ; FC(F)(F)C1(c2ccc(SSc3ccc(C4(C(F)(F)F)N=N4)cc3)cc2)N=N1]

### Synthesis of S-(4-(3-(trifluoromethyl)-3H-diazirin-3-yl)phenyl) 4-(3-(trifluoromethyl)-3H-diazirin-3-yl)benzothioate (UDC2)

A 250 mL round-bottom flask was charged with 4-(3-(trifluoromethyl)-3H-diazirin-3-yl)benzenethiol (1.00 g, 4.58 mmol, 1.0 equiv) and anhydrous dichloromethane (57 mL). NaHCO₃ (481 mg, 5.73 mmol, 1.25 equiv) was added in one portion and the resulting suspension was stirred at room temperature for 15 min. Iodine (1.74 g, 6.87 mmol, 1.5 equiv) was added portion was and the resulting dark purple mixture was stirred for 40 min. Half-saturated aqueous sodium thiosulfate (50 mL) was added and the biphasic mixture was stirred vigorously until all excess iodine had been consumed as evidenced by full discoloration of the organic phase (approximately 30 min). The phases were separated, and the aqueous phase was extracted with dichloromethane. The combined organic phases were washed with brine, dried over Na₂SO₄, filtered and concentrated. Purification by flash column chromatography (hexanes) furnished the title product (886 mg, 2.29 mmol, 89%) as a pale-yellow oil (melting point ˜20° C.). R_{f} (hexanes): 0.39; ¹H NMR (500 MHZ, CDCl₃) δ: 7.50-7.45 (4H, m, H₅), 7.13 (4H, d, J=8.3 Hz, H₄); ¹³C NMR (126 MHz, CDCl₃) δ: 138.8 (2C, C₆), 128.3 (2C, C₃), 127.4 (4C, C₄), 127.2 (4C, C₅), 122.1 (2C, q, ¹J=274.7 Hz, C₁), 28.4 (q, 2J=40.8 Hz, C₂), ¹⁹F NMR (471 MHZ, CDCl₃) δ: −64.3; HRMS ASAP+ found [M+H]⁺ 435.0166, C₁₆H₈F₆N₄S₂⁺ requires 435.0173; IR (film) vₘₐₓ: 2990, 1715, 1341, 1181, 1148, 936, 814, 696.

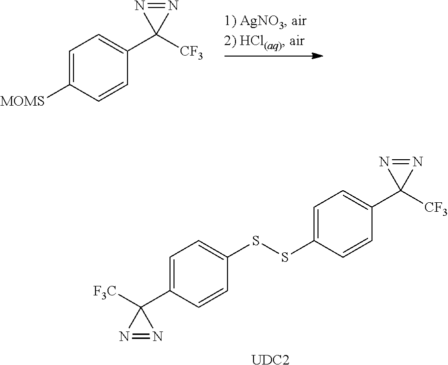

### Synthesis of UDC2 from 3-(4-((methoxymethyl)thio)phenyl)-3-(trifluoromethyl)-3H-diazirine

A solution of 3-(4-((methoxymethyl)thio)phenyl)-3-(trifluoromethyl)-3H-diazirine (2.00 g, 7.63 mmol, 1.0 equiv) in anhydrous ethanol (240 mL) was stirred open to air at 55° C. Silver nitrate (1.55 g, 9.15 mmol, 1.2 equiv) was added in one portion with vigorous stirring. The mixture was stirred at 55° C. for 2 h, during which a white precipitate was formed, rendering the reaction highly viscous. All volatiles were removed in vacuo using a rotary evaporator. 6 M aqueous hydrochloric acid (218 mL) was added to the crude mixture and the reaction was stirred at ambient temperature open to air for 1.5 h. Dichloromethane (100 mL) was added and stirring was continued for 1 h. The biphasic mixture was filtered and the layers were separated. The aqueous layer was extracted with dichloromethane (2×100 mL). The combined organic phases were dried over Na₂SO₄ filtered and concentrated in vacuo. Purification by silica flash column chromatography (elution: hexanes) yielded the title product (1.36 g, 3.13 mmol, 82%) as a volatile pale-yellow oil that solidified at lower temperatures. Analytical data were identical to those reported above for UDC2.

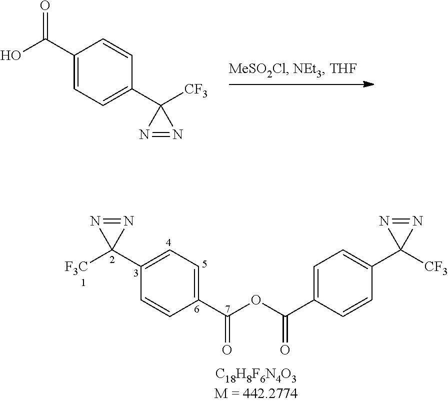 [SMILES: O=C(O)c1ccc(C2(C(F)(F)F)N=N2)cc1 ; O=C(OC(=O)c1ccc(C2(C(F)(F)F)N=N2)cc1)c1ccc(C2(C(F)(F)F)N=N2)cc1]

### Synthesis of 4-(3-(trifluoromethyl)-3H-diazirin-3-yl)benzoic anhydride (UDC3)

A solution of 4-(3-(trifluoromethyl)-3H-diazirin-3-yl)benzoic acid (1.50 g, 6.52 mmol, 1.0 equiv) in anhydrous THF (20 mL) was cooled to 0° C., and MeSO₂Cl (277 μL, 3.58 mmol, 0.55 equiv) was added dropwise. The resulting pale-yellow solution was stirred for 5 min at 0° C. before triethylamine (1.55 mL, 11.2 mmol, 1.7 equiv) was added dropwise resulting in the formation of a white precipitate. After stirring for 1 h at 0° C., the ice bath was removed and the reaction mixture was allowed to warm to room temperature for 10 min. The mixture was concentrated (rotary evaporation at 40° C.) and directly purified by column chromatography (10% dichloromethane in hexanes containing 1% (v/v) HCl (2 M in Et₂O), to 25% dichloromethane in hexanes containing 1% (v/v) HCl (2 M in Et₂O)) to give the title compound as a white, crystalline solid (1.38 g, 3.12 mmol, 96%). R_{f} (25% dichloromethane in hexanes+1% (v/v) HCl (2 M in Et₂O)): 0.41; ¹H NMR (500 MHZ, CDCl₃) δ: 8.16 (4H, d, J=8.6 Hz, H₅), 7.13 (4H, d, J=8.3 Hz, H₄); ¹³C NMR (126 MHz, CDCl₃) δ: 161.1 (2C, C₇), 135.9, (2C, C₆), 131.0 (4C, C₅), 129.7 (2C, C₃), 127.0 (4C, C₄), 121.9 (2C, q, J=274.8 Hz, C₁), 28.6 (2C, q, J=41.0 Hz, C₂); ¹⁹F NMR (471 MHz, CDCl₃) δ: −64.8; HRMS ESI+ found [M+Na]⁺ 463.0416, C₁₈H₈F₆N₄NaO₃⁺ requires 465.0398; IR (film) vₘₐₓ: 2991, 2898, 2677, 1688, 1346, 1290, 1202, 1184, 1153, 942, 736. Note: UDC3 was found to be stable towards ambient moisture and even aqueous workup. However, UDC3 hydrolyzes readily on neutral or basic silica. Hence, acidification of silica prior to and during column chromatography is required in order to obtain high yields. Commercially available solutions of HCl in diethyl ether or dioxane were found to furnish the best results. Acidification using acetic or formic acid leads to undesired cross-anhydride products.

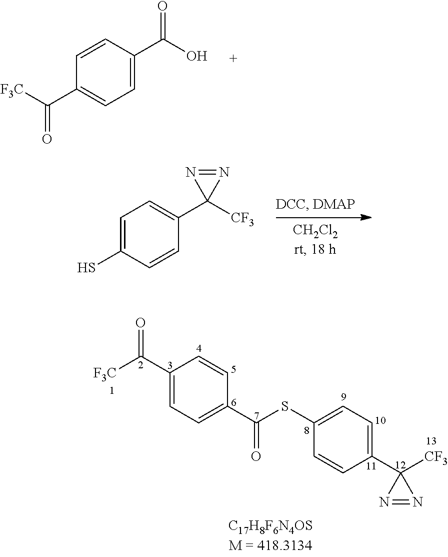 [SMILES: O=C(O)c1ccc(C(=O)C(F)(F)F)cc1 ; FC(F)(F)C1(c2ccc(S)cc2)N=N1 ; O=C(Sc1ccc(C2(C(F)(F)F)N=N2)cc1)c1ccc(C(=O)C(F)(F)F)cc1]

### Synthesis of S-(4-(3-(trifluoromethyl)-3H-diazirin-3-yl)phenyl) 4-(2,2,2-trifluoroacetyl)benzothioate

A 50 mL Schlenk flask was charged with 4-(2,2,2-trifluoroacetyl)benzoic acid (960 mg, 4.40 mmol, 1.2 equiv), DCC (1.06 g, 5.13 mmol, 1.4 equiv) and DMAP (202 mg, 1.65 mmol, 0.45 equiv), sealed and backfilled with nitrogen (3×). A solution of 4-(3-(trifluoromethyl)-3H-diazirin-3-yl)benzenethiol (800 mg, 3.67 mmol, 1.0 equiv) in anhydrous dichloromethane (13 mL) was added via syringe, and the resulting mixture stirred at room temperature for 18 h. The reaction mixture was filtered through celite, the filter cake washed with diethyl ether, and the filtrate concentrated in vacuo. The residue was purified by silica flash column chromatography (gradient elution: hexanes then 10% dichloromethane in hexanes), yielding the title product (1.02 g, 2.44 mmol, 67%) as a colorless oil R_{f} (10% diethyl ether in hexanes): 0.53; ¹H NMR (500 MHZ, CDCl₃) δ 8.19 (2H, d, J=8.4 Hz, H_{4/5}), 8.17-8.13 (2H, m, H_{4/5}), 7.61-7.54 (2H, m, H₁₀), 7.29 (2H, d, J=8.2 Hz, H₉); ¹³C NMR (126 MHz, CDCl₃) δ 188.5 (C₇), 180.00 (q, J=36.0 Hz, C₂), 141.4 (C₆), 135.3 (2C, C₉), 133.7 (C₃), 131.1 (C_{8/11}), 130.6 (2C, q, J=2.0 Hz, C₁₀), 128.7 (C_{8/11}), 128.1 (2C, C₅), 127.4 (2C, C₄), 122.06 (q, J=274.7 Hz, C₁₂), 116.5 (q, J=291.0 Hz, C₁), 28.5 (q, J=40.7 Hz, C₁₂); ¹⁹F NMR (471 MHz, CDCl₃) δ: −64.1 (s, F₃C-1), —70.9 (s, F₃C-13).

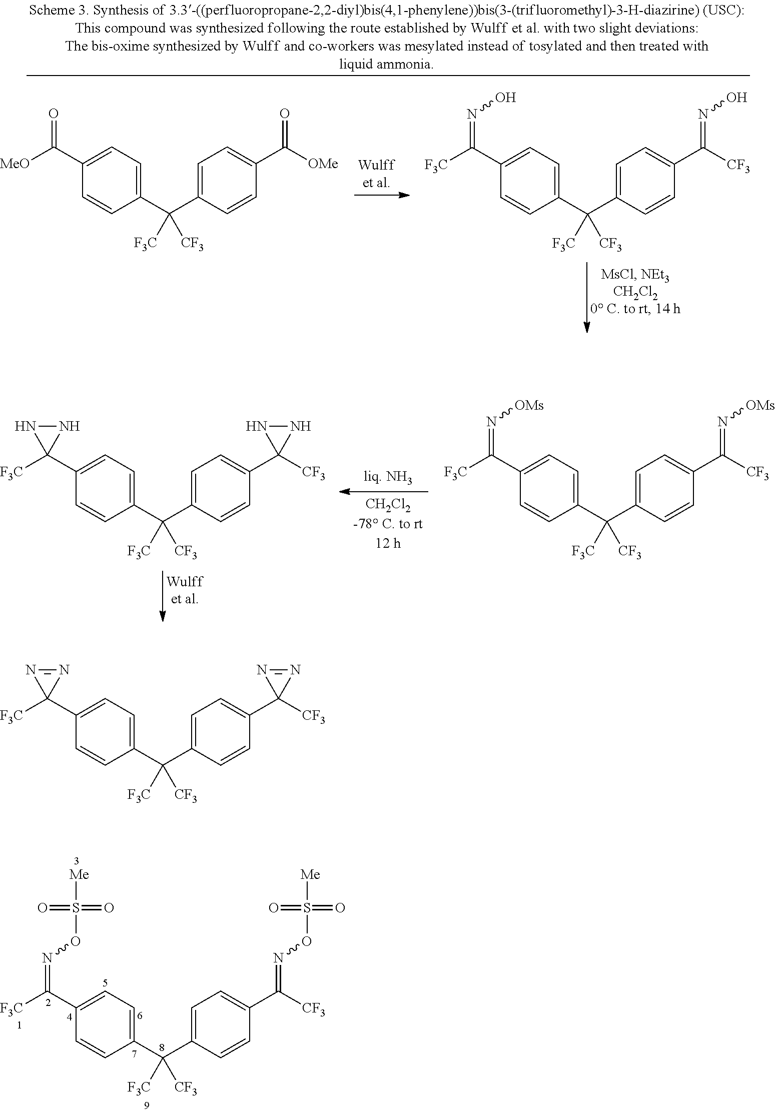 [SMILES: COC(=O)c1ccc(C(c2ccc(C(=O)OC)cc2)(C(F)(F)F)C(F)(F)F)cc1 ; ON=C(c1ccc(C(c2ccc(C(=NO)C(F)(F)F)cc2)(C(F)(F)F)C(F)(F)F)cc1)C(F)(F)F ; FC(F)(F)C1(c2ccc(C(c3ccc(C4(C(F)(F)F)NN4)cc3)(C(F)(F)F)C(F)(F)F)cc2)NN1 ; CS(=O)(=O)ON=C(c1ccc(C(c2ccc(C(=NOS(C)(=O)=O)C(F)(F)F)cc2)(C(F)(F)F)C(F)(F)F)cc1)C(F)(F)F ; FC(F)(F)C1(c2ccc(C(c3ccc(C4(C(F)(F)F)N=N4)cc3)(C(F)(F)F)C(F)(F)F)cc2)N=N1 ; CS(=O)(=O)ON=C(c1ccc(C(c2ccc(C(=NOS(C)(=O)=O)C(F)(F)F)cc2)(C(F)(F)F)C(F)(F)F)cc1)C(F)(F)F]

### Synthesis of 1,1′-((perfluoropropane-2,2-diyl)bis(4,1-phenylene))bis(2,2,2-trifluoroethan-1-one) O,O-dimethylsulfonyl dioxime

A solution of 1,1′-((perfluoropropane-2,2-diyl)bis(4,1-phenylene))bis(2,2,2-trifluoroethan-1-one) dioxime (4.89 g, 9.29 mmol, 1.0 equiv) in anhydrous dichloromethane (48 mL) was cooled to 0° C., triethylamine (4.14 mL, 29.7 mmol, 3.2 equiv) was added dropwise followed by MsCl (1.58 mL, 20.4 mmol, 2.2 equiv). The reaction was allowed to warm to room temperature overnight (approximately 15 h) and then quenched by addition of sat. aq. NH₄Cl (50 mL). Water (15 mL) was added to dissolve the resulting precipitate. The mixture was extracted with diethyl ether (3×50 ml). The combined organic phases were dried over Na₂SO₄, filtered, and concentrated. The resulting crude solid was purified by silica flash column chromatography (gradient elution: hexanes to 25% ethyl acetate in hexanes), yielding the title product (4.83 g, 7.08 mmol, 76%) as a colorless solid.

The title compound was isolated as an 18:1 mixture of diastereomers. Only signals corresponding to the major diastereomer are reported: R_{f} (25% ethyl acetate in hexanes): 0.23; 1H NMR (500 MHZ, acetone) δ 7.83 (4H, d, J=8.7 Hz, H₅), 7.71 (4H, d, J=8.3 Hz, H₆), 3.46 (6H, s, H₂); ¹³C NMR (126 MHZ, acetone) δ: 153.8 (2C, q, J=33.8 Hz, C₂), 136.8 (2C, C₄), 131.6 (4C, C₅), 130.1 (4C, C₆), 127.0 (2C, C₇), 124.8 (2C, q, J=288.1 Hz, C₉), 120.7 (2C, q, J=276.5 Hz, C₁), 65.6 (app q, J=51.8 Hz, C₈), 37.3 (2C, C₃); ¹⁹F NMR (471 MHz, acetone) δ −63.1 (s, F₃C-9), −66.7 (s, F₃C-1); HRMS ESI+ found [M+Na]⁺ 705.0001, C₂₁H₁₄F₁₂N₂NaO₆S₂⁺ requires 704.9994 IR (film) vₘₐₓ: 3030, 1702, 1381, 1249, 1187, 1142, 971, 892, 814, 756, 674, 518, 487.

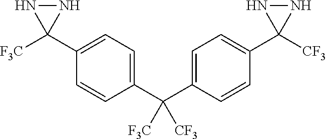 [SMILES: FC(F)(F)C1(c2ccc(C(c3ccc(C4(C(F)(F)F)NN4)cc3)(C(F)(F)F)C(F)(F)F)cc2)NN1]

### Synthesis of 3,3′-((perfluoropropane-2,2-diyl)bis(4,1-phenylene))bis(3-(trifluoromethyl) diaziridine)

Anhydrous ammonia was condensed (approx. 40 mL) into a pre-cooled Schlenk flask at −78° C. using a dry-ice acetone cold finger. To the stirred liquid ammonia, a solution of 1,1′-((perfluoropropane-2,2-diyl)bis(4,1-phenylene))bis(2,2,2-trifluoroethan-1-one) O,O-dimethyl sulfonyl dioxime (4.80 g, 7.03 mmol, 1.0 equiv) in anhydrous tetrahydrofuran (40 mL) was added dropwise at −78° C. The solution was stirred for 1 h at −78° C. and then gradually warmed to room temperature over the course of 15 h, while allowing excess ammonia to vent. Nitrogen was bubbled through the resulting suspension for 10 min to remove residual ammonia. The resulting suspension was concentrated, and dichloromethane (200 mL) was added. The resulting mixture was washed with sat. aq. NH₄Cl (150 mL). The phases were separated, and the aqueous phase was extracted with dichloromethane (2×100 mL). The combined organic phases were dried over Na₂SO₄, filtered, and concentrated. The resulting crude solid was purified by silica flash column chromatography (gradient elution: 50% dichloromethane in hexanes to dichloromethane to 10% ethyl acetate in dichloromethane), yielding the title product (3.03 g, 5.78 mmol, 82%) as a colorless solid.

### Example 3. Modeled Carbene Activation and Insertion Crosslinking on Cyclohexane by UDC1, 2, and 3

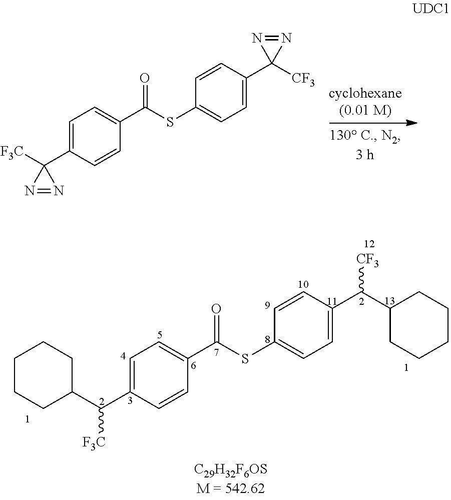 [SMILES: O=C(Sc1ccc(C2(C(F)(F)F)N=N2)cc1)c1ccc(C2(C(F)(F)F)N=N2)cc1 ; O=C(Sc1ccc(C(C2CCCCC2)C(F)(F)F)cc1)c1ccc(C(C2CCCCC2)C(F)(F)F)cc1]

### Thermal Protocol:

A pressure tube was charged with UDC1 (90 mg, 0.21 mmol) under nitrogen and cyclohexane (21 ml) was added. The tube was sealed and heated to 130° C. (careful: gas evolution, overpressure!) for 3 h. The reaction vessel was allowed to cool to room temperature and the reaction mixture was concentrated. The crude reaction mixture was purified by column chromatography (12% dichloromethane in hexanes, to 20% dichloromethane in hexanes containing to give the title compound as a colorless oil (11.2 mg, 20.6 μmol, 10%) as a 1:1 mixture of diastereomers.

A similar experiment was carried out using UDC1 (12.9 mg, 30.0 μmol) in 3.0 mL cyclohexane at 130° C. for 3 h. After cooling to room temperature, 1,4-dinitirobenzene was added as an internal standard for quantification. The crude reaction mixture was concentrated and then analyzed by ¹H- and ¹⁹F-NMR spectroscopy. A yield of 3.3 mg (6.07 μmol, 20%) of the crosslinked product was determined. ¹H NMR (500 MHZ, CDCl₃) δ 8.03-7.99 (2H, m, H₅), 7.50-7.47 (2H, m, H₄), 7.37 (2H, d, J=8.3 Hz, H_{9/10}), 7.33 (2H, d, J=8.3 Hz, H_{9/10}), 3.19-3.04 (2H, m, H₂), 2.07-1.91 (4H, m, H_{1/13}), 1.83-1.74 (2H, m, H_{1/13}), 1.69-1.60 (4H, m, H_{11/13}), 1.52 (1H, d, J=13.5 Hz, H_{1/13}), 1.45 (1H, d, J=13.8 Hz, H_{1/13}), 1.37-1.27 (m, 2H, H_{1/13}), 1.21-1.06 (6H, m, H_{1/13}), 0.92-0.74 (2H, m, H₁); ¹³C NMR (126 MHz, CDCl₃) δ 189.6 (C₇), 141.5 (q, ³J=2.2 Hz, C₃), 137.0 (q, ³J=2.2 Hz, C₁₁), 136.1 (C₆), 135.1 (C₅), 130.2 (C_{4/10}), 129.8 (C_{4/10}), 127.8 (C₉), 127.1 (q, ¹J=281.6 Hz), 126.9 (q, ¹J=281.6 Hz), 126.9 (C₅), 56.7-55.8 (m, 2C, C₁₃), 38.7 (C₁), 38.7 (C₁), 31.6 (C₁), 30.9 (C₁), 30.9 (C₁), 29.9 (C₁), 26.3 (C₁), 26.2 (C₁), 26.13 (C₁), 26.08 (C₁), 26.0 (C₁); ¹⁹F NMR (471 MHz, CDCl₃) δ −62.17 (d, J=9.8 Hz), −62.27 (d, J=9.9 Hz); HRMS ASAP+ found [M+H]⁺ 543.2170, C₂₉H₃₃OSF₆⁺ requires 543.2156.

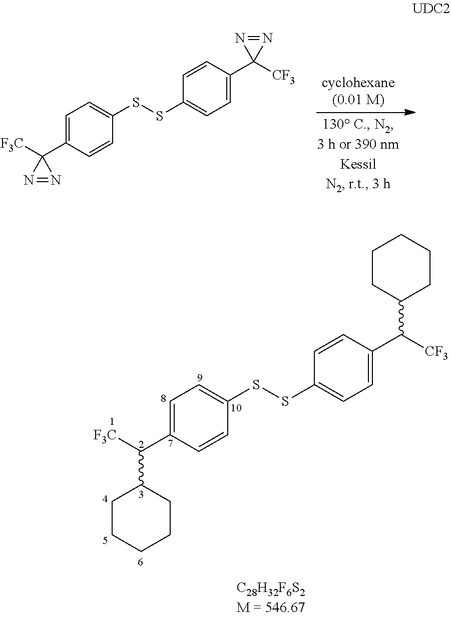 [SMILES: FC(F)(F)C1(c2ccc(SSc3ccc(C4(C(F)(F)F)N=N4)cc3)cc2)N=N1 ; FC(F)(F)C(c1ccc(SSc2ccc(C(C3CCCCC3)C(F)(F)F)cc2)cc1)C1CCCCC1]

### UV-Irradiation Protocol:

A 1.5 dram vial was charged with UDC2 (17.4 mg, 40.0 μmol) under nitrogen and cyclohexane (4 ml) was added. The vial was sealed tightly and irradiated with a 390 nm Kessil lamp for 3 h. The reaction mixture was concentrated and 1,4-dinitirobenzene was added as an internal standard for quantification. The crude reaction mixture was then analyzed by ¹H- and ¹⁹F-NMR spectroscopy.

To obtain isolable quantities of the C—H insertion product, the protocol outlined above was set up 8 times in parallel (139 mg, 320 μmol of UDC2 in total). Upon completion of the reactions, the crude mixtures were combined, concentrated, and purified by flash column chromatography (hexanes) to give the desired addition product (19.1 mg, 34.9 μmol, 11%) as a colorless oil and a 1:1 mixture of diastereomers. R_{f} (hexanes): 0.61; ¹H NMR (500 MHZ, CDCl₃) δ 7.52-7.38 (4H, m, H₉), 7.23-7.14 (4H, m, H₈), 3.02 (2H, p, J=10.1 Hz, H₂), 2.04-1.88 (4H, m), 1.81-1.72 (2H, m), 1.68-1.58 (4H, m), 1.47 (2H, d, J=12.7 Hz), 1.36-1.24 (2H, m), 1.21-1.03 (6H, m), 0.80 (2H, qd, J=12.1, 3.4 Hz); ¹³C NMR (126 MHz, CDCl₃) δ 136.7 (4C, C₁₀), 134.5 (2C, q, J=2.7 Hz, C₇), 130.1 (4C, C₉), 127.5 (4C, C₈), 127.17 (2C, q, J=281.5 Hz, C₁), 55.9 (2C, q, J=25.1 Hz, C₂), 38.7 (2C, C₃), 31.6 (2C, C₄), 30.8 (2C, C₄′), 26.3 (2C, C_{5/6}), 26.2 (2C, C_{5/6}), 26.1 (2C, C_{5/6}); ¹⁹F NMR (471 MHz, CDCl₃) δ −62.45, −62.47; HRMS ASAP+ found [M+H]⁺ 547.1914, C₂₈H₃₃S₂F₆⁺ requires 547.1928; IR (film) vₘₐₓ: 2926, 2854, 1493, 1251, 1135, 1098, 813, 731, 518.

### Thermal Protocol:

A pressure tube was charged with (8.2 mg, 20.0 μmol) under nitrogen and cyclohexane (2 ml) was added. The tube was sealed and heated to 130° C. (careful: gas evolution, overpressure!) for 3 h. The reaction vessel was allowed to cool to room temperature and the crude mixture was concentrated. 1,4-Dinitirobenzene was added as an internal standard for quantification and the crude reaction mixture was then analyzed by ¹H- and ¹⁹F-NMR spectroscopy.

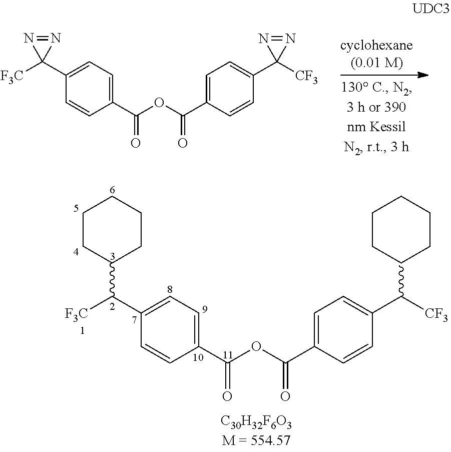 [SMILES: O=C(OC(=O)c1ccc(C2(C(F)(F)F)N=N2)cc1)c1ccc(C2(C(F)(F)F)N=N2)cc1 ; O=C(OC(=O)c1ccc(C(C2CCCCC2)C(F)(F)F)cc1)c1ccc(C(C2CCCCC2)C(F)(F)F)cc1]

### UV-Irradiation Protocol:

A 1.5 dram vial was charged with UDC3 (8.9 mg, 20.0 μmol) under nitrogen and cyclohexane (2 mL) was added. The vial was sealed tightly and irradiated with a 390 nm Kessil lamp for 3 h. The reaction mixture was concentrated and 1,4-dinitirobenzene was added as an internal standard for quantification. The crude reaction mixture was then analyzed by ¹H- and ¹⁹F-NMR spectroscopy.

Thermal Protocol: A pressure tube was charged with (102 mg, 230.0 μmol) under nitrogen and cyclohexane (23 ml) was added. The tube was sealed and heated to 130° C. (careful: gas evolution, overpressure!) for 3 h. The reaction vessel was allowed to cool to room temperature and the crude mixture was concentrated. The crude reaction mixture was purified by column chromatography (1% ethyl acetate in hexanes containing 1% (v/v) HCl (2 M in Et₂O), to 2.5% ethyl acetate in hexanes containing 1% (v/v) HCl (2 M in Et₂O)) to give the title compound as a colorless oil (9.1 mg, 21.0 μmol, 9%) as a 1:1 mixture of diastereomers. R_{f} (3% ethyl acetate in hexanes+1% (v/v) HCl (2 M in Et₂O)): 0.36; ¹H NMR (500 MHZ, CDCl₃) δ 8.16-8.11 (4H, m, H₉), 7.41 (4H, d, J=8.3 Hz, H₈), 3.17 (2H, p, J=9.8 Hz, H₂), 2.06-1.95 (4H, m), 1.83-1.75 (2H, m), 1.68-1.60 (4H, m), 1.48-1.41 (2H, m), 1.32 (2H, qt, J=12.5, 2.9 Hz), 1.22-1.05 (6H, m), 0.83 (2H, qd, J=12.4, 3.4 Hz); ¹³C NMR (126 MHz, CDCl₃) δ 162.0 (2C, Cu), 142.49 (2C, q, J=2.8 Hz, C₇), 130.9 (4C, C₉), 129.9 (4C, C₈), 128.5 (2C, C₁₀), 126.9 (2C, q, J=281.6 Hz, C₁), 56.4 (2C, q, J=25.1 Hz, C₂), 38.7 (2C, C₃), 31.6 (2C, C₄), 30.9 (2C, C₄′), 26.2 (2C, C_{5/6}), 26.1 (2C, C_{5/6}), 26.0 (2C, C_{5/6}); ¹⁹F NMR (471 MHz, CDCl₃) δ −62.12, −62.14; HRMS ESI+ found [M+Na]⁺ 577.2161, C₃₀H₃₂O₃NaF₆⁺ requires 577.2153; IR (film) vₘₐₓ: 2934, 2857, 1790, 1728, 1612, 1252, 1221, 1003, 707.

### Example 4. Molecular Trials for Blend Compatibilization and Mixing

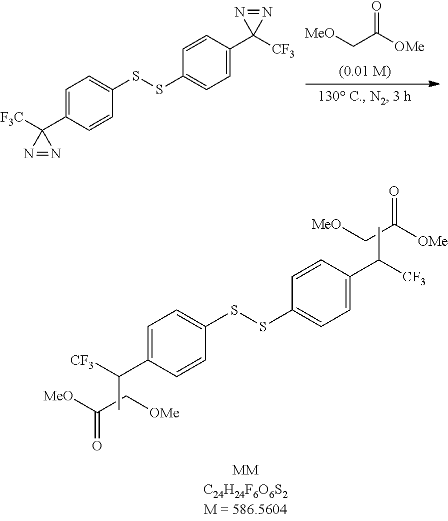

### Crosslinking of Methyl Methoxyacetate by UDC2 (Reference Compound):

A pressure tube was charged with (17.4 mg, 40.0 μmol) of UDC2 under nitrogen and methyl methoxyacetate (4.0 ml) was added. The tube was sealed and heated to 130° C. (careful: gas evolution, overpressure!) for 3 h. The reaction vessel was allowed to cool to room temperature and the crude mixture was concentrated. The crude reaction mixture consisted of a complex mixture of regio- and diastereomers. The crude mixture was purified by preparative thin layer chromatography (30% ethyl acetate in hexanes). All fractions were analyzed by ¹H-, ¹⁹F-NMR, and HRMS. The desired product (MM) was unambiguously identified by HRMS: ASAP+ found [M]⁺ 586.0913, C₂₄H₂₄O₆S₂F₆⁺ requires 586.0919.

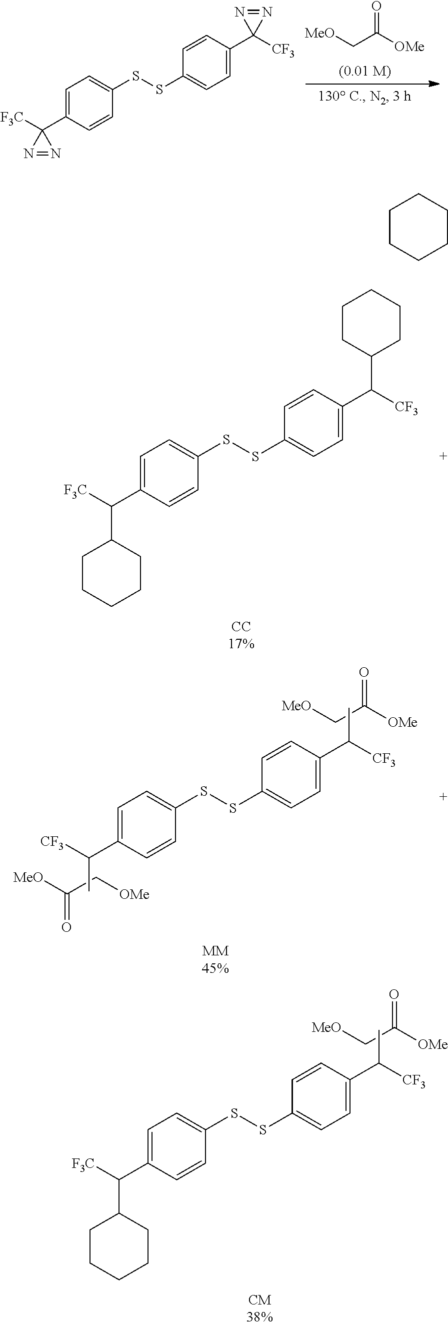

### Crosslinking of Cyclohexane/Methyl Methoxyacetate by UDC2:

A pressure tube was charged with (17.4 mg, 40.0 μmol) of UDC2 under nitrogen. Cyclohexane (2.0 mL) and methyl methoxyacetate (4.0 ml) were added. The tube was sealed and heated to 130° C. (careful: gas evolution, overpressure!) for 3 h. The reaction vessel was allowed to cool to room temperature and the crude mixture was concentrated. The crude mixture was purified by preparative thin layer chromatography (20% ethyl acetate in hexanes). All fractions were analyzed by ¹H-, ¹⁹F-NMR, and HRMS. The fractions containing the mixed-crosslinked product (CM) were unambiguously identified by HRMS: ASAP+ found [M]⁺ 566.1389, C₂₆H₂₈O₃S₂F₆⁺ requires 566.1384.

The reaction protocol outlined above was repeated and the crude reaction mixture was analyzed by SFC-MS using authentic samples of CC, MM, and CM (see above) as reference compounds. (SFC conditions: Water BEH (Ethylene Bridged Hybrid) column, gradient elution, 0% MeCN to 5% MeCN in CO₂ over 10 min, 1.2 ml/min, 40° C.). The following relative yields were determined: CC: 17%, MM: 45% and CM: 38%.

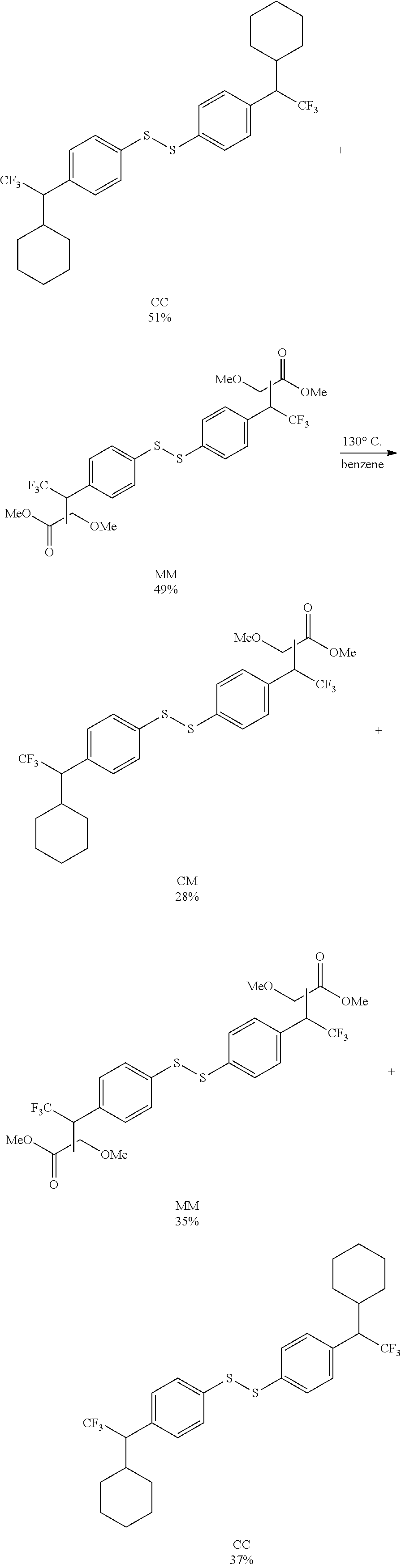

### Disulfide Exchange Experiments:

A small pressure tube was charged with CC (5.0 mg, 9.15 μmol) and MM (5.6 mg, 9.15 μmol) benzene (0.1 mL, 0.09 M) was added under argon. The vessel was sealed and heated to 130° C. for 24 h. The reaction mixture was concentrated and analyzed by SFC-MS. The following relative product distribution was determined: CC: 37%, MM: 35% and CM: 28%.

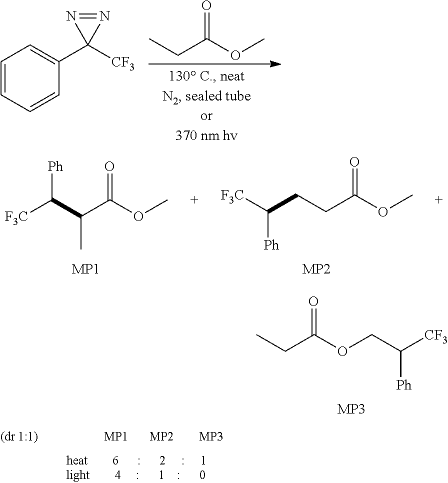

### Determination of C—H Insertion Selectivity for Polyesters:

Methyl propionate was used as a model substrate to investigate the regioselectivity of the C—H insertion of aryl-trifluoro diazirines into polyesters. A small pressure tube was charged with commercially available 3-phenyl-3-(trifluoromethyl)-3H-diazirine (7.2 μL, 7.5 mg, 40.0 μmol) and methyl propionate. (4.0 mL, 0.01 M). The vessel was sealed and heated to 130° C. for 3 h. The reaction mixture was cooled to room temperature and the volatiles were removed by rotary evaporation. The thus obtained crude mixture was analyzed by ¹H-NMR spectroscopy. MP1 and MP2 are known compounds. An authentic sample of MP3 was obtained through chemical synthesis (vide infra). Using NMR spectroscopic data and reference spectra, a ratio of MP1:MP2:MP3 of 6:2:1 was determined. This agrees with earlier reports concluding preferential insertion of phenyl-trifluoro diazirine-derived carbenes into α-carbonyl C—H bonds of ethyl acetate.

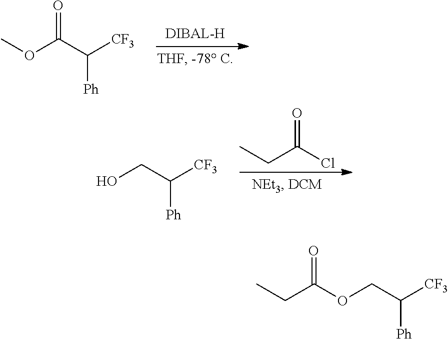

### Synthesis of 3,3,3-trifluoro-2-phenylpropyl propionate (MP3, Reference Compound)

Methyl 3,3,3-trifluoro-2-phenylpropanoate (150 mg, 0.67 mmol, 1.0 equiv.) was dissolved in THF (5 mL) under nitrogen and cooled to −78° C. DIBAL-H (1.0 M in DCM, 3.30 mL, 3.30 mmol, 5.0 equiv.) was added dropwise. The reaction mixture was allowed to stir at room temperature for 3 h and then cooled to −78° C. MeOH (0.48 mL) was added dropwise followed by aq. NaOH (1 M, 0.42 mL). The mixture was allowed to warm to room temperature and stirred overnight. The thus obtained suspension was filtered through Celite® to obtain crude 3,3,3-trifluoro-2-phenylpropan-1-ol which was directly used in the subsequent step.

A 10 mL flask was charged with crude 3,3,3-trifluoro-2-phenylpropan-1-ol (126 mg, 0.66 mmol, 1.0 equiv.), CH₂Cl₂ (3.3 mL, 0.2 M), and triethylamine (120 μL, 0.86 mmol, 1.3 equiv.). The reaction mixture was cooled to 0° C. and a solution of propionyl chloride (88 μL, 0.99 mmol, 1.5 equiv.) in CH₂Cl₂ (0.6 mL) was added dropwise. The mixture was stirred at 0° C. for 40 min and then allowed to warm to room temperature overnight. The reaction was quenched by addition of sat. aq. NaHCO₃ (6.6 mL) and diluted with CH₂Cl₂ (6.6 mL). The layers were separated and the aqueous phase was extracted with CH₂Cl₂ (2×6.6 mL). The combined organic phases were dried over MgSO₄, filtered and concentrated. The crude product was purified by flash column chromatography (5% EtOAc in hexanes) to give 3,3,3-trifluoro-2-phenylpropyl propionate (55.8 mg, 34%) as a colorless oil. ¹H NMR (500 MHZ, CD₂Cl₂) δ 7.43-7.37 (m, 3H), 7.35-7.31 (m, 2H), 4.58 (dd, J=11.6, 6.8 Hz, 1H), 4.50-4.43 (m, 1H), 3.75 (qt, J=9.2, 6.9 Hz, 1H), 2.33-2.22 (m, 2H), 1.05 (t, J=7.6 Hz, 3H); ¹³C NMR (126 MHZ, CD₂Cl₂) δ 174.10, 132.61, 129.42, 129.22, 129.09, 126.41 (q, J=280.0 Hz), 62.03 (d, J=3.1 Hz), 49.53 (q, J=26.6 Hz), 27.69, 9.10; ¹⁹F NMR (470 MHz, CD₂Cl₂) δ −68.07 (d, J=9.2 Hz).

### Example 5. Crosslinking Methods

### General Polymer Crosslinking Procedures/Considerations (Method 1):

Typical polymer crosslinking by UDC1, 2, and 3 was performed on the dissolved target polymer in a suitable solvent (toluene or CHCl₃) at or below 85° C. in a round bottom flask. Selected UDC (5 wt. %) was added to the polymer mixture in a toluene solution and allowed to mix briefly before being removed from heat and cooled in an ice bath (0° C.). Careful monitoring of temperature is critical to avoiding pre-mature UDC activation. Solvent was removed under vacuum, which yielded a white powder that was placed in a vacuum oven at 50° C. until a constant mass was achieved. The powder was moved into an oven at 125° C. for 3 h, during which time the polymer was crosslinked by activated carbenes, visibly confirmed by powder yellow tint. The following strategy, based on discussed thermal/solubility/performance results among similar literature precedent, was deemed effective for homogeneous dispersion of and crosslink by UDC. This procedure was the full requirement for UDCs 2 and 3. UDC1 CANs were further diluted (swelled) in heated (85° C.) toluene for 4 h. Then 3% base catalyst (DBU) and free tetra-thiol source PETMP (equimolar to UDC) was added to the mixture and stirred briefly, after which the sample was cooled using an ice bath (0° C.) and the toluene was removed under vacuum at 60° C. Polymer-UDC1 was then dried in a 60° C. vacuum oven overnight or until a constant mass was achieved. Dried samples were stored in sealed containers at ambient conditions. Small-scale initial trials were performed at 50 mg polymer loading, while large-scale trials were performed at 5.0 g polymer loading.

### Homopolymer Small-Scale Crosslinking Procedure (0, 5, 10 wt. % UDC) for Polyesters: PLA, P3HB:

The specified polyester (50 mg) was mixed with chloroform (15 mL) in a 20 mL glass scintillation vial and placed in an 85° C. automatic heating and orbiting shaker until fully dissolved. Upon full or near-full solubility, a solution of selected UDC (2.5 mg) dissolved in chloroform (1 mL) was added quantitatively to the stirring mixture. The UDC was briefly mixed into the polymer solution before cooling over an ice bath (0° C.), which yielded a white gelatinous precipitate. Solvents were removed (50° C.) by rotovap, and the sample was left to dry in a vacuum oven (60° C.) overnight or until a constant mass was achieved. The obtained white powder was moved to a 125° C. oven for 3 h to crosslink via thermal activation of UDC carbene precursors. Crosslinked polymers appeared light yellow, a visual signal of successful installment.

Procedure for Polyolefins LMWPE, LDPE, HDPE, UHMWPE, it-PP:

The specified polymer (50 mg) was mixed with toluene (15 mL) in a 20 mL glass scintillation vial and placed in an 85° C. automatic heating and orbiting shaker until fully dissolved. Upon full solubility, a solution of selected UDC (2.5 mg) dissolved in toluene (1 mL) was added quantitatively to the stirring mixture. The UDC is briefly mixed into the polymer solution before cooling over an ice bath (0° C.), which yielded a white gelatinous precipitate. Solvents were removed (60° C.) by rotovap, and the sample was left to dry in a vacuum oven (60° C.) overnight or until a constant mass was achieved. The obtained white powder was moved to a 125° C. oven for 3 h to crosslink via thermal activation of UDC carbene precursors. Crosslinked polymers appeared light yellow, a visual sign of successful installment.

### Homopolymer Crosslinking Procedure (5 wt. % UDC) for Polymers HDPE, LDPE, LMWPE:

A 500 mL round bottom flask was charged with toluene (100 mL), specified polymer powder or pellets (5.0 g), and a stir bar. The mixture was heated to 85° C. and allowed to stir until partial or full solubility was observed, at which point selected UDC (0.25 g, 5.0 wt. %) in a separate toluene solution (5 mL) was added into the solution. (Note: PMMA and others may not require heating to achieve solubility). The mixture was stirred briefly before cooling over an ice bath (0° C.), which yielded a gelatinous white precipitate. Toluene was then removed under vacuum at 60° C. and the precipitated UDC-imbedded-polymer was dried under vacuum at 60° C. overnight or until a constant mass was achieved. The resulting white powder was then placed in a 125° C. oven for 3 h to crosslink via thermal activation of UDC carbenes. Crosslinked polymers appeared light yellow, a visual sign of successful installment.

### 50/50 wt. % Polyolefin/Polyester Blend Small-Scale Crosslinking Procedure for Polymers LMWPE, LDPE, PLA, and P3HB:

In a 20 mL glass scintillation vial polyethylene (25 mg) was mixed with toluene (10 mL) and placed in an 85° C. automatic heating and orbiting shaker until fully dissolved. Separately, a 20 mL scintillation vial was charged with the specified polyester (25 mg, 50 wt. %), 10 mL of chloroform, and a stir bar and mixed until full solubility was observed. The polyester solution was added to the heated toluene mixture, and allowed to mix for 30 minutes, at which time a solution of selected UDC (2.5 mg) dissolved in toluene (1 mL) was added quantitatively to the stirring mixture. The solution blend of all three components was briefly mixed before cooling over an ice bath (0° C.), which yielded a white gelatinous precipitate. Solvents were removed (60° C.) by rotovap, and the sample was left to dry in a vacuum oven (60° C.) overnight or until a constant mass was achieved. The obtained white powder was moved to a 125° C. oven for 3 h to crosslink via thermal activation of UDC carbene precursors. Crosslinked blends appeared light yellow, a visual sign of successful installment.

### 50/50 wt. % Polyolefin/Polyester Blend Crosslinking Procedure for Polymers LMWPE, LDPE, PLLA, and P3HB:

In a 250 mL round bottom flask polyethylene (2.5 g) was vigorously stirred in toluene (100 mL) at 85° C. until full solubility was observed. In a separate 250 mL round bottom flask, specified PLLA or P3HB pellets (2.5 g, 50 wt. %) were vigorously stirred in chloroform (50 mL) until full solubility was observed. The polyester solution was then quickly added to the polyethylene solution. Additional stirring at 85° C. was carried out for 30 min, which achieved stable solution-blended polymers. Selected UDC (0.25 g) was then dissolved in toluene (5 mL) and added quantitatively to the stirring mixture. The UDC-blend was stirred briefly before cooling over an ice bath (0° C.), which yielded a white gelatinous precipitate. Solvents were removed under vacuum at 60° C. and the received UDC-blend was dried under vacuum at 60° C. overnight or until a constant mass was achieved. The white powder was moved to a 125° C. oven for 3 h to crosslink via thermal activation of UDC carbene precursors. Crosslinked blends appeared light yellow, a visual sign of successful installment. Virgin (0%) blends of LMWPE and PLLA/P3HB were prepared by the above procedures, without the steps of UDC addition.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A crosslinking agent of Formula I:
R—X-L-G-L-X—R (I),
wherein,
each L is independently an aryl, heteroaryl, or alkylene;
G is anhydride, disulfide, thioester, ester, boronic ester, thioboronic ester, silyl ether, siloxane, imine, or urethane;
each R is independently alkyl, aryl or heteroaryl; and
X is C(N)₂ or carbene;
wherein each alkyl, alkylene, aryl, or heteroaryl is optionally substituted.

2. The crosslinking agent of claim 1 wherein L is phenyl.

3. The crosslinking agent of claim 2 wherein X is in the para-position relative to G.

4. The crosslinking agent of claim 1 wherein R is CF₃.

5. The crosslinking agent of claim 1 wherein X is

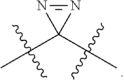 [SMILES: *C1(*)N=N1]

6. The crosslinking agent of claim 1 wherein G is —(C═O)O(O═C)—, —S—S—, or —S(C—O)—.

7. The crosslinking agent of claim 1 wherein each R—X-L- group consists of the same moieties.

8. The crosslinking agent of claim 1 wherein Formula I is represented by Formula II:

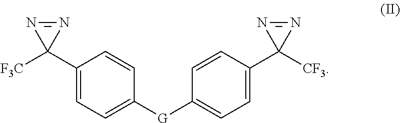

9. The crosslinking agent of claim 1 wherein the crosslinking agent is:

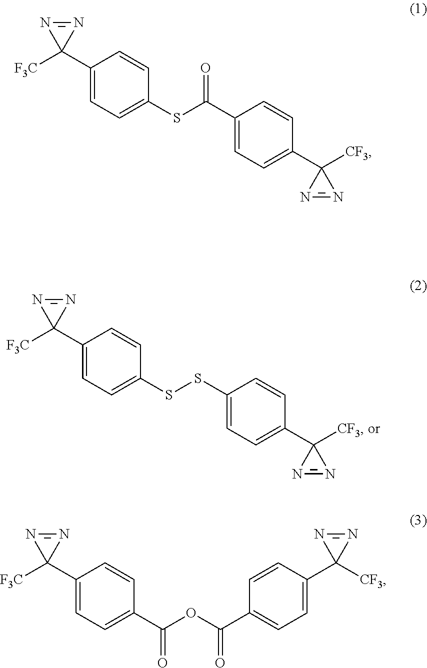

10. A dynamic thermoset that is crosslinked with the crosslinking agent of claim 1.

11. The dynamic thermoset of claim 10 wherein the dynamic thermoset comprises about 0.1 wt. % to about 15 wt. % of a crosslinker, wherein the crosslinker is a moiety of the crosslinking agent that is covalently bonded at its carbene carbon atoms to polymers in the dynamic thermoset.

12. The dynamic thermoset of claim 10 wherein the dynamic thermoset comprises an apolar polyolefin, a polar polyester, or a combination thereof.

13. The dynamic thermoset of claim 10 wherein the dynamic thermoset comprises low-molecular-weight polyethylene (LMWPE), low-density polyethylene (LDPE), high-density polyethylene (HDPE), ultra-high-molecular-weight polyethylene (UHMWPE), isotactic polypropylene (ifPP), polystyrene (PS), poly(L-lactic acid) (PLLA), poly(3-hydroxybutyrate) (P3HB), or a combination thereof.

14. A method for crosslinking mixed plastics, comprising:
a) forming a mixture comprising apolar polymers, polar polymers, or a combination thereof;
b) blending the mixture and the crosslinking agent of claim 1 to form a blend; and
c) activating the crosslinking agent;
wherein the mixed plastics are thereby crosslinked.

15. The method of claim 14 wherein the mixture is blended with about 0.1 wt. % to about 15 wt. % of the crosslinking agent, wherein the wt. % is based on the combined weight of the apolar polymer and the polar polymer.
